(19) United States
(12) Patent Application Publication
(10) Pub. No.: US 2024/0424100 A1
(43) Pub. Date: Dec. 26, 2024

(54) METHODS OF TREATING LUPUS NEPHRITIS USING ANTI-CD19 CAR-T CELL THERAPIES

(71) Applicant: Kyverna Therapeutics, Inc., Emeryville, CA (US)

(72) Inventors: Dominique C. Borie, Palo Alto, CA (US); James Chung, Thousand Oaks, CA (US); Tom Van Blarcom, Oakland, CA (US); Ranjita Sengupta, Belmont, CA (US); Karen Walker, Mukilteo, WA (US); Justin Chou, Los Angeles, CA (US)

(21) Appl. No.: 18/827,623

(22) Filed: Sep. 6, 2024

Related U.S. Application Data
(63) Continuation of application No. PCT/US2023/077760, filed on Oct. 25, 2023.
(60) Provisional application No. 63/587,329, filed on Oct. 2, 2023.
(60) Provisional application No. 63/518,745, filed on Aug. 10, 2023.
(60) Provisional application No. 63/512,061, filed on Jul. 5, 2023.
(60) Provisional application No. 63/419,193, filed on Oct. 25, 2022.

(30) Foreign Application Priority Data
Jan. 3, 2023 (WO) PCT/US2023/010034

(51) Int. Cl. A61K 39/00; A61P 37/06; C07K 14/725; C07K 16/28

(52) U.S. Cl. CPC A61K 39/464412; A61K 39/4611; A61K 39/4631; A61P 37/06; C07K 14/7051; C07K 16/2803; C07K 2317/622

(57) ABSTRACT

Provided herein are methods and compositions for treating a subject having an B cell-associated autoimmune disease, such as lupus nephritis, using T cells engineered with a chimeric antigen receptor that binds CD19.

### RELATED APPLICATIONS

This application is a continuation of International Application No. PCT/US2023/077760 filed on Oct. 25, 2023, which claims the benefit of and priority to U.S. Provisional Patent Application No. 63/419,193, filed on Oct. 25, 2022; International (PCT) Application No. PCT/US2023/010034, filed on Jan. 3, 2023; U.S. Provisional Patent Application No. 63/512,061, filed on Jul. 5, 2023; U.S. Provisional Patent Application No. 63/518,745, filed on Aug. 10, 2023; and U.S. Provisional Patent Application No. 63/587,329, filed on Oct. 2, 2023, the entire contents of each of which are incorporated by reference herein for all purposes.

### SEQUENCE LISTING

This application contains a Sequence Listing which has been submitted electronically in XML format and is hereby incorporated by reference in its entirety. The XML copy, created on Feb. 13, 2024, is titled KVN-008USWOC1_SL.xml and is 51,179 bytes in size.

### TECHNICAL FIELD

The present disclosure generally relates to T cells engineered to express chimeric antigen receptors (CARs) (e.g., fully human anti-CD19 CARs) and their use in the treatment and/or prevention of autoimmune diseases, such as lupus nephritis.

### BACKGROUND

Treatment of autoimmune diseases generally requires multiple treatments to control the disease. The most common treatments are corticosteroids and cytotoxic drugs, which can be very toxic. These drugs can also suppress the entire immune system, resulting in serious infection, and can have adverse side effects on bone marrow, liver, and/or kidneys. Thus, there are limitations to their chronic use and of the use of multiple agents. Most patients develop severe manifestations of disease that may be life-threatening and require close monitoring and active treatment.

Current therapeutic regimens for B cell based autoimmune diseases, e.g., lupus nephritis, include high-dose glucocorticoids combined with immunosuppressants such as cyclophosphamide (CYC) or rituximab in order to induce remission and prevent further organ damage, however relapse is common and remains a significant clinical management challenge. Upon disease control, lower doses of glucocorticoids and a broader range of immunosuppressants such as azathioprine (AZA), methotrexate, and mycophenolate mofetil (MMF) are used to maintain disease control. Rituximab or CYC may be repeated upon relapse of disease activity. However, despite these treatments, significant unmet need remains for new therapies that can readily achieve sustained remission, effectively address the frequent relapses with existing therapies, and reduce the significant background immunosuppressive therapies required to maintain good disease control.

### SUMMARY

Provided herein are methods and compositions for treating a subject with an autoimmune disease or disorder. Notably, provided herein are methods and compositions for treating a subject with an autoimmune disease or disorder for which conventional therapeutic regimens have been shown to be ineffective (e.g., due to their toxicity profiles or limitations in their pharmacological action). In some embodiments, conventional therapeutic regimens (i.e., standard of care (SOC) therapy) are withdrawn so that patients are free of any supportive immunomodulatory drugs for a duration of time (e.g., at least 1 week, 2 weeks, 3 weeks, 4 weeks, 1 month, 2 months, 3 months, 4 months, 6 months, 8 months, 1 year, 2 years, 3 years, or more) after receiving provided methods and compositions. In some embodiments, conventional therapeutic regimens (i.e., SOC therapy) are withdrawn ahead of collecting host cells (e.g., lymphocytes, such as T cells) to be engineered by any method described herein (e.g., at least 1 day, 2 days, 3 days, 4 days, 5 days, 6 days, 7 days, 8 days, 9 days, 10 day, 11 days, 12 days, 13 days, 14 days, 3 weeks, 4 weeks, or more before collecting host cells). Further provided herein are methods and compositions for treating a subject with an autoimmune disease or disorder, where said methods and compositions are used in place of known conventional therapeutic regimens. The present disclosure further provides for methods and compositions for treating a subject with a B cell-associated autoimmune disease or disorder. In some embodiments, a B cell-associated autoimmune disease or disorder includes systemic lupus erythematosus. In some embodiments, a B cell-associated autoimmune disease is lupus nephritis. In some embodiments, a B cell-associated autoimmune disease is Class III or Class IV lupus nephritis. In some embodiments, a B cell-associated autoimmune disease is Class III lupus nephritis. In some embodiments, a B cell-associated autoimmune disease is Class IV lupus nephritis. In some embodiments, a B cell-associated autoimmune disease is Class II lupus nephritis. In some embodiments, the present disclosure provides for methods and compositions that result in elimination of B cells at tissue sites normally not accessible to conventional treatments.

The methods of the present disclosure use T cells engineered to express anti-CD19 CAR constructs, which can reduce or deplete B cells responsible for a patient's autoimmune disease or disorder. In many embodiments, the anti-CD19 CAR constructs have a lower toxicity profile as compared to conventional treatment. In some embodiments, an anti-CD19 CAR is substantially non-toxic to a subject receiving treatment with the CAR therapy. In some embodiments, such low toxicity or non-toxic CAR therapies provided by the present disclosure allow for higher doses and/or multiple doses which result in depletion of B cells at sites not treatable with conventional autoimmune treatments due to their toxicity profile. Indeed, CAR therapies provided herein remarkably exhibit low levels of toxicity commonly associated with CAR therapy (e.g., anti-CD19 CAR therapy), including cytokine-release syndrome (CRS) and neurologic toxicities. In some embodiments, the present disclosure provides methods of and compositions for treating a B-cell associated autoimmune disease or disorder (e.g., lupus nephritis, such as Class II, Class III or Class IV lupus nephritis) with an anti-CD19 CAR. In many embodiments of any of the provided methods and compositions described herein, an anti-CD19 CAR comprises a fully human chimeric antigen receptor comprising an extracellular antigen-binding domain, a transmembrane domain, and an intracellular domain, wherein the intracellular domain comprises a cytoplasmic signaling domain and one or more co-stimulatory domains. See, e.g., U.S. Pat. No. 10,287,350, which is incorporated by reference herein in its entirety.

The present disclosure provides, among other things, a method of treating lupus nephritis (e.g., Class II, Class III or Class IV lupus nephritis), the method comprising administering to a subject in need thereof a therapeutically effective amount of T cells that comprises a vector comprising a nucleic acid encoding a chimeric antigen receptor (CAR), wherein the CAR comprises, from N-terminus to C-terminus: (a) an antigen-binding fragment of an anti-CD19 antibody; (b) a transmembrane domain; and (c) an intracellular T cell signaling domain from human CD3ζ.

In some embodiments, an anti-CD19 antibody is a human antibody.

In some embodiments, an antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 1, 2, and 3, respectively, and the light chain variable domain comprising CDR1, CDR2, and CDR3 amino acid sequences of SEQ ID NOs: 4, 5, and 6, respectively.

In some embodiments, an antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 25, 26, and 3, respectively, and the light chain variable domain comprising CDR1, CDR2, and CDR3 amino acid sequences of SEQ ID NOs: 4, 5, and 6, respectively.

In some embodiments, a heavy chain variable domain comprises an amino acid sequence at least 90% identical to SEQ ID NO: 7, and the light chain variable domain comprises an amino acid sequence at least 90% identical to SEQ ID NO: 8. In some embodiments, a heavy chain variable domain comprises the amino acid sequence of SEQ ID NO: 7, and the light chain variable domain comprises the amino acid sequence of SEQ ID NO: 8.

In some embodiments, an antigen-binding fragment of the anti-CD19 antibody comprises the amino acid sequence of SEQ ID NO: 17.

In some embodiments, a transmembrane domain is from human CD8.

In some embodiments, a transmembrane domain comprises an amino acid sequence at least 90% identical to SEQ ID NO: 11. In some embodiments, a transmembrane domain comprises the amino acid sequence of SEQ ID NO: 11.

In some embodiments, an intracellular T cell signaling domain from human CD3ζ comprises an amino acid sequence at least 90% identical to SEQ ID NO: 23. In some embodiments, an intracellular T cell signaling domain from human CD3ζ comprises the amino acid sequence of SEQ ID NO: 23.

In some embodiments, a CAR further comprises an intracellular T cell signaling domain from human CD28. In some embodiments, an intracellular T cell signaling domain from human CD28 comprises the amino acid sequence of SEQ ID NO: 21.

In some embodiments, a CAR does not comprise an intracellular T cell signaling domain from 4-1BB.

In some embodiments, a CAR comprises an amino acid sequence of SEQ ID NO: 10 or 13.

In some embodiments, a vector is a lentivirus vector. In some embodiments, a vector further comprises a murine stem cell virus (MSCV) U3 promoter operably linked to the nucleic acid.

In some embodiments, at least 10% (e.g., at least 15%, at least 20%, at least 25%, at least 30%, at least 40%, at least 50%, at least 60%, at least 70%, at least 80%, at least 90%, at least 95%, or at least 99%) of the provided T cells express a CAR (e.g., any CAR provided herein). In some embodiments, provided T cells comprise at least 10% (e.g., at least 15%, at least 20%, at least 25%, at least 30%, at least 40%) of CD8+ cytotoxic T cells. In some embodiments, provided T cells comprise at least 10% (e.g., at least 15%, at least 20%, at least 25%, at least 30%, at least 40%, at least 50%) of CD4+ helper T cells.

In some embodiments, the lupus nephritis is active, biopsy-proven, proliferative lupus nephritis of Class III or Class IV according to the 2018 ISN/RPS criteria. In some embodiments, the lupus nephritis is active, biopsy-proven, proliferative lupus nephritis of Class III according to the 2018 ISN/RPS criteria. In some embodiments, the lupus nephritis is active, biopsy-proven, proliferative lupus nephritis of Class IV according to the 2018 ISN/RPS criteria. In some embodiments, the lupus nephritis is active, biopsy-proven, proliferative lupus nephritis of Class II according to the 2018 ISN/RPS criteria.

In some embodiments, the subject tests positive for anti-nuclear antibodies, e.g., has an anti-nuclear antibody titer greater than or equal to 1:80, as determined by an Hep-2 immunofluorescence assay or an enzyme immunoassay assay. In some embodiments, the subject tests positive for anti-dsDNA antibodies, e.g., has an anti-dsDNA antibody concentration greater than or equal to 30 IU/mL, as determined by an enzyme-linked immunosorbent assay. In some embodiments, the subject tests positive for an anti-Smith antibody. In some embodiments, the subject tests positive for anti-phospholipid antibodies. In some embodiments, the subjects tests positive for anti-phospholipids antibodies and experiences clinical manifestations consistent with an anti-phospholipid syndrome.

In some embodiments, a therapeutically effective dose is in a range of about 5×10⁷ to 1×10⁸, about 5×10⁷ to 9×10⁷, about 5×10⁷ to 8×10⁷, about 5×10⁷ to 7×10⁷, about 5×10⁷ to 6×10⁷, about 6×10⁷ to 1×10⁸, about 7×10⁷ to 1×10⁸, about 8×10⁷ to 1×10⁸, about 9×10⁷ to 1×10⁸, about 6×10⁷ to 9×10⁷, or about 7×10⁷ to 8×10⁷ of the T cells. In some embodiments, a therapeutically effective dose is in a range of about 5×10⁷ to 1×10⁸ of the T cells. In some embodiments, a therapeutically effective dose is about 5×10⁷ of the T cells (e.g., 5×10⁷ of the T cells). In some embodiments, a therapeutically effective dose is about 1×10⁸ of the T cells (e.g., 1×10⁸ of the T cells).

In some embodiments, provided T cells are administered by intravenous infusion.

In some embodiments, the subject receives a single dose of the T cells.

In some embodiments, the subject has received a lymphodepletion treatment. In some embodiments, lymphodepletion treatment comprises intravenous administration of cyclophosphamide (e.g., at a dose of 300 mg/m²) and of fludarabine (e.g., at a dose of 30 mg/m²) prior to administration of the T cells, e.g., once every day for 3 days, starting 5 to 7 days prior to administration of the T cells. In some embodiments, the subject does not receive a lymphodepletion treatment prior to administration of the T cells. In some embodiments, the subject has received a minimized lymphodepletion treatment. In some embodiments, a minimized lymphodepletion treatment comprises intravenous administration of cyclophosphamide (e.g., at a dose of 150 mg/m²) and of fludarabine (e.g., at a dose of 15 mg/m²) prior to administration of the T cells, e.g., once every day for 3 days, starting 5 to 7 days prior to administration of the T cells. In some embodiments, a minimized lymphodepletion treatment reduces lymphocytes in a subject by about 30%, 35%, 40%, 45%, 50%, 55%, 60%, 65%, or 70% relative to the subject before receiving lymphodepletion treatment or another suitable control. In some embodiments, the subject has received a minimized lymphodepletion treatment resulting in about 50% reduction of lymphocytes in the subject relative to the subject before receiving lymphodepletion treatment or another suitable control.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

Unless otherwise defined, all technical and scientific terms used herein have the same meaning as commonly understood by one of ordinary skill in the art to which this disclosure pertains. Although methods and materials similar or equivalent to those described herein can be used in accordance with the present disclosure, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a series of graphs showing cytotoxic activity of Systemic Lupus Erythematosus (SLE) patient-derived peripheral blood mononuclear cells (PBMCs) transduced with a Hu19-CD828Z chimeric antigen receptor (CAR) construct against NALM6 (CD19⁺) cells.

FIG. 2 is a series of graphs showing cytotoxic activity of SLE patient- and Healthy Donor Derived-PBMCs transduced with a Hu19-CD828Z CAR construct against autologous primary B cells expressing CD19.

FIGS. 3A-3B are graphs showing interferon-gamma (IFNK) release by SLE patient-derived PBMCs transduced with a Hu19-CD828Z CAR construct following co-culture with NALM6 (CD19⁺) tumor cells (FIG. 3A) or with autologous primary B cells expressing CD19 (FIG. 3B).

FIGS. 4A-4C are graphs showing proliferation of Hu19-CD828Z transduced PBMCs following co-culture with NALM6 (CD19⁺) tumor cells (FIG. 4A), autologous primary B cells expressing CD19 (FIG. 4B), or control K562 (CD19⁻) cells (FIG. 4C).

FIG. 5 is a schematic of a lentivirus vector encoding an anti-CD19 CAR transgene.

### DETAILED DESCRIPTION

### Definitions

“About” a number, as used herein, refers to range including the number and ranging from 10% below that number to 10% above that number. “About” a range refers to 10% below the lower limit of the range, spanning to 10% above the upper limit of the range.

As used herein, the term “antibody” refers to any immunoglobulin, whether naturally occurring or wholly or partially synthetically produced. All derivatives thereof which maintain specific binding ability are also included in the term. In some embodiments, the term “antibody” refers to any protein having a binding domain which is homologous or largely homologous to an immunoglobulin-binding domain. Antibody proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. In certain embodiments, an antibody may be a member of the IgG immunoglobulin class.

As used herein, “derived from” or “derivative” indicates a structural similarity and a functional similarity between a subject molecule and a reference molecule (e.g., between polynucleotides, polypeptides, etc.). With respect to structural similarity, the subject molecule does not necessarily comprise the same sequence (e.g., nucleic acid sequence, amino acid sequence, etc.) as the reference molecule, but has at least 75%, 80%, 85%, 90%, 91%, 92%, 93%, 94%, 95%, 96%, 97%, 98%, 99% or higher identity to the sequence (e.g., nucleic acid sequence, amino acid sequence, etc.) of the reference molecule or a fragment thereof, the fragment comprising at least 10%, 20%, 30%, 40%, 50%, 60%, 70%, 80%, 90%, 91%, 92%, 93%, 94%, 95%, 96%, 97%, 98%, 99%, or more of the sequence of the reference molecule. With respect to functional similarity, the subject molecule has at least 1%, 5%, 10%, 20%, 30%, 40%, 50%, 60%, 70%, 80%, 90%, 95%, 96%, 97%, 98%, 99% or more of an activity of the reference molecule or the fragment thereof as determined in a suitable assay. For example, a subject polypeptide may be considered to be derived from a reference polypeptide when the subject polypeptide has structural similarity, as defined above, to the reference polypeptide and retains certain function(s), such as certain intermolecular or intramolecular interactions (e.g., binding to a protein, e.g., a particular receptor, or a signaling activity), though such interactions could be stronger, equivalent, or weaker than that of the reference polypeptide. As another non-limiting example, a subject polynucleotide may be considered to be derived from a reference polynucleotide when the subject polynucleotide has structural similarity to the reference polynucleotide, as defined above, and encodes a protein or protein fragment that is a derivative of the protein encoded by the reference polynucleotide, or has the same or similar function (e.g., as a regulatory element, e.g., promoter or enhancer) as the reference polynucleotide. Functional similarity takes into account the context of the disclosure. For example, when applied to a subject intracellular T cell signaling domain derived from a reference protein (e.g., CD3ζ, CD28), the subject intracellular T cell signaling domain has structural and functional similarities to an intracellular T cell signaling domain of the reference protein as known in the art. Similarly, when applied to a subject transmembrane domains derived from a reference protein, the subject transmembrane domain has structural and functional similarities to a transmembrane domain of the reference protein as known in the art. In one non-limiting example, an intracellular T cell signaling domain derived from a CD3ζ molecule retains sufficient CD3ζ structure such that it has the ability to transduce a signal (e.g., ZAP-70 activation) under appropriate conditions.

As used herein, the term “functional fragment” of a reference biomolecule, e.g., a polynucleotide or polypeptide, refers to a shorter and/or smaller derivative of the reference biomolecule that has at least 75%, 80%, 85%, 90%, 91%, 92%, 93%, 94%, 95%, 96%, 97%, 98%, 99% or more sequence identity to the sequence of a fragment of the reference biomolecule.

As used herein the term “operably linked” refers to polynucleotide sequences placed into a functional relationship with one another. For instance, a promoter or enhancer is operably linked to a coding sequence if it regulates, or contributes to modulation of, the transcription of a coding sequence. Operably linked DNA sequences encoding regulatory sequences are typically contiguous to a coding sequence. However, enhancers can function when separated from a promoter by up to several kilobases or more. Additionally, multi-cistronic constructs can include multiple coding sequences which use only one promoter by including a 2A self-cleaving peptide, an IRES element, etc. Accordingly, some polynucleotide elements may be operably linked but not contiguous.

As used herein, the term “patient” or “subject” are used interchangeably to refer to any organism to which a compositions disclosed herein may be administered, e.g., for experimental, diagnostic, prophylactic, and/or therapeutic purposes. Typical patients include animals (e.g., mammals such as mice, rats, rabbits, non-human primates, and/or humans). In some embodiments, a patient or subject is a human. In many embodiments, a patient is a human subject suffering from an autoimmune disease or disorder.

### Methods and Compositions for Treating Autoimmune Diseases

The present disclosure provides methods and compositions that can be used to treat a subject identified as having an autoimmune disease. The present disclosure further provides methods and compositions for treating B cell-associated autoimmune diseases. In some embodiments, a B cell-associated autoimmune disease is systemic lupus erythematosus. In some embodiments, a B cell-associated autoimmune disease is lupus nephritis. In some embodiments, a B cell-associated autoimmune disease is Class III or Class IV lupus nephritis. In some embodiments, a B cell-associated autoimmune disease is Class III lupus nephritis. In some embodiments, a B cell-associated autoimmune disease is Class IV lupus nephritis. In some embodiments, a B cell-associated autoimmune disease is Class II lupus nephritis.

The present disclosure provides, among other things, methods and compositions for reducing the number of B cells in a tissue in a subject having an autoimmune disease. The present disclosure also provides methods and compositions for treating a subject having an autoimmune disease. Additionally, the present disclosure provides engineered T cells (e.g., T cells engineered to express any CAR described herein) and method of making engineered T cells. The present disclosure also provides engineered nucleic acids (e.g., engineered nucleic acid constructs) that encode engineered polypeptides (e.g., any engineered polypeptide described herein, e.g., a CAR).

The present disclosure appreciates that B cells express a wide array of cell surface molecules during their differentiation and proliferation, e.g., CD19. CD19 is widely expressed on B cells during all phases of B cell development from pro-B cells to plasmablasts. The present disclosure further appreciates, that because of the ubiquity of CD19 on B cells, CD19 can function as a therapeutic target for certain provided methods and compositions (e.g., methods and compositions for treating an autoimmune disease). Accordingly, in some embodiments, provided herein are methods and compositions for reducing the number of B cells in a subject (e.g., in a tissue of a subject) via targeting of CD19. In some embodiments, the present disclosure provides for methods and compositions for treating a subject having a B cell-associated autoimmune disease via targeting of CD19. In some embodiments, the present disclosure provides for engineered T cells that target CD19. In some embodiments, the present disclosure provides for engineered nucleic acids that express one or more polypeptides that target CD19. In many embodiments of the present disclosure a CAR that binds to CD19 is used to target cells that express CD19 (e.g., B cells).

### Chimeric Antigen Receptors

In some embodiments, a chimeric antigen receptor (CAR) of the present disclosure comprises an extracellular domain, a transmembrane domain, and an intracellular domain. In some embodiments, an extracellular domain is or comprises an antigen-binding domain (e.g., a CD19 binding domain, such as an anti-CD19 scFv). In some embodiments, a transmembrane domain is or comprises a transmembrane domain or functional fragment thereof derived from any suitable cell membrane-associated polypeptide, e.g., obtained from a membrane-binding polypeptide or transmembrane polypeptide. In some embodiments, a transmembrane domain is or comprises a transmembrane domain or functional fragment thereof derived from a T cell receptor alpha chain, a T cell receptor beta chain, a CD3 zeta chain, a CD28 polypeptide, or a CD8 polypeptide (e.g., a CD8α polypeptide). In some embodiments, an intracellular domain is or comprises an intracellular signaling domain (e.g., any of intracellular signaling domains described herein, e.g., derived from a CD28 or CD3 polypeptide). In some embodiments, an intracellular signaling domain comprises one or more signaling sequences or motifs. In some embodiments, one or more signaling sequences, or signaling motifs, are essential for the functional signaling capacity of a polypeptide (e.g., an intracellular signaling domain). In some embodiments, a signaling sequence is a sequence derived from a CD3 polypeptide (e.g., a CD3 zeta polypeptide). In some embodiments, a signaling sequence is derived from a CD28 polypeptide. In some embodiments, a signaling sequence is or comprises a co-stimulatory domain (e.g., any co-stimulatory domain described herein, e.g., derived from a CD28 polypeptide). In some embodiments, a CAR of the present disclosure is a human CAR.

### Extracellular Domain

In some embodiments, an extracellular domain used in accordance with the present disclosure comprises an antigen-binding domain (e.g., any antigen-binding domain described herein). In some embodiments, an antigen-binding domain is or comprises an antibody sequence (e.g., an immunoglobulin) or antigen-binding fragment thereof (e.g., any antibody or antigen-binding fragment thereof described herein). Anticalins or other alternative scaffolds are also contemplated.

In some embodiments, the antigen-binding domain comprises one or more Fab, Fab′, F(ab′)₂, Fv, domain antibody (dAb), single-chain antibody (scFv), chimeric antibody, diabody, triabody, tetrabody, scAb, or single domain antibody (e.g., V_{H}H or V_{NAR}) polypeptide sequences. In some embodiments, the antigen-binding domain comprises at least a portion of an immunoglobulin that is sufficient to confer specific antigen-binding to a polypeptide (e.g., an antibody fragment comprising an antigen-binding portion). In some embodiments, the antigen-binding domain comprises an scFv. In some embodiments, the scFv comprises a VH and VL domain of an antibody. In some embodiments, the scFv comprises a spacer sequence between the VH and the VL. In some embodiments, the scFv comprises a spacer sequence as set forth in SEQ ID NO: 9 between the VH and the VL. In some embodiments, the antigen-binding domain is humanized, or fully human (e.g., derived from a suitable human polypeptide). Exemplary methods of generating fully human antibodies are described in Lu et al., (2020) J. Biomed. Sci. (2020) 27(1):1.

In some embodiments, the antigen-binding domain binds to a target antigen (e.g., a polypeptide). In some embodiments, the antigen-binding domain binds specifically to a target antigen (e.g., a polypeptide). In some embodiments, the antigen-binding domain binds to a CD19 polypeptide (e.g., a CD19 polypeptide present at the surface of a cell, e.g., a B cell). In some embodiments, the antigen-binding domain binds specifically to a CD19 polypeptide. In some embodiments, the antigen-binding domain comprises an antibody, or antigen-binding fragment thereof, that binds to a CD19 polypeptide. In some embodiments, the antigen-binding domain comprises a scFv sequence that binds to a CD19 polypeptide (e.g., an anti-CD19 scFv).

It is generally understood that CD19 expression is largely restricted to B lymphocytes. CD19 has two N-terminal extracellular Ig-like domains separated by a non-Ig-like domain, a hydrophobic transmembrane domain, and a large C-terminal cytoplasmic domain. The CD19 protein forms a complex with several membrane proteins including complement receptor type 2 (CD21) and tetraspanin (CD81) and this complex reduces the threshold for antigen-initiated B cell activation. Activation of this B-cell antigen receptor complex activates the phosphatidylinositol 3-kinase signaling pathway and the subsequent release of intracellular stores of calcium ions. An example of a human CD19 polypeptide sequence includes, without limitation, NCBI reference sequence: NP_001171569.1, and fragments and derivatives thereof.

In some embodiments, an antigen-binding domain comprises a variable region of an anti-CD19 antibody. In some embodiments, an antigen-binding domain comprises a variable region of an anti-CD19 monoclonal antibody. In some embodiments, an antigen-binding domain comprises a variable region of a mouse or human anti-CD19 monoclonal antibody. An anti-CD19 monoclonal antibody can be obtained or derived from a subject (e.g., a mouse, a rat, a rabbit, a human, etc.) using any suitable method. In some embodiments, an antigen-binding domain comprises a light chain variable region and a heavy chain variable region of a mouse, human, or humanized anti-CD19 monoclonal antibody. In some embodiments, an antigen-binding domain comprises a light chain variable region of a mouse, human, or humanized anti-CD19 monoclonal antibody. In some embodiments, an antigen-binding domain comprises a heavy chain variable region of a mouse, human, or humanized anti-CD19 monoclonal antibody. The 47G4 antibody (described in U.S. Patent Application Publication No. 2010/0104509, which is incorporated herein by reference in its entirety) is one example of a human anti-CD19 monoclonal antibody that can be used in accordance with the present disclosure.

In some embodiments, the antigen-binding domain that binds CD19 comprises a heavy chain variable domain and a light chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 1, SEQ ID NO: 2, and SEQ ID NO: 3, respectively, and the light chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 4, SEQ ID NO: 5, and SEQ ID NO: 6, respectively. In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NO: 1, SEQ ID NO: 2, and SEQ ID NO: 3, respectively, and the light chain variable domain comprising CDR1, CDR2, and CDR3 amino acid sequences as set forth in SEQ ID NO: 4, SEQ ID NO: 5, and SEQ ID NO: 6, respectively.

In some embodiments, the antigen-binding domain that binds CD19 comprises a heavy chain variable domain and a light chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 25, SEQ ID NO: 26, and SEQ ID NO: 3, respectively, and the light chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 4, SEQ ID NO: 5, and SEQ ID NO: 6, respectively. In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NO: 25, SEQ ID NO: 26, and SEQ ID NO: 3, respectively, and the light chain variable domain comprising CDR1, CDR2, and CDR3 amino acid sequences as set forth in SEQ ID NO: 4, SEQ ID NO: 5, and SEQ ID NO: 6, respectively.

In some embodiments, the antigen-binding domain that binds CD19 comprises a heavy chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 1, SEQ ID NO: 2, and SEQ ID NO: 3, respectively. In some embodiments, the antigen-binding domain that binds CD19 comprises a heavy chain variable domain, the heavy chain variable domain comprising CDR1, CDR2, and CDR3 amino acid sequences as set forth in SEQ ID NO: 1, SEQ ID NO: 2, and SEQ ID NO: 3, respectively.

In some embodiments, the antigen-binding domain that binds CD19 comprises a heavy chain variable domain, the heavy chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 25, SEQ ID NO: 26, and SEQ ID NO: 3, respectively. In some embodiments, the antigen-binding domain that binds CD19 comprises a heavy chain variable domain, the heavy chain variable domain comprising CDR1, CDR2, and CDR3 amino acid sequences as set forth in SEQ ID NO: 25, SEQ ID NO: 26, and SEQ ID NO: 3, respectively.

In some embodiments, the antigen-binding domain that binds CD19 comprises a light chain variable domain, the light chain variable domain comprising CDR1, CDR2, and CDR3 amino acid sequences having at least 80%, at least 85%, at least 90%, at least 95%, at least 96%, at least 97%, at least 98%, at least 99%, or greater sequence identity to SEQ ID NO: 4, SEQ ID NO: 5, and SEQ ID NO: 6, respectively. In some embodiments, the antigen-binding domain that binds CD19 comprises a light chain variable domain, the light chain variable domain comprising CDR1, CDR2, and CDR3 amino acid sequences as set forth in SEQ ID NO: 4, SEQ ID NO: 5, and SEQ ID NO: 6, respectively.

In some embodiments, the antigen-binding domain that binds CD19 comprises a heavy chain variable domain and a light chain variable domain, the heavy chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 7, and the light chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 8. In some embodiments, the antigen-binding domain that binds CD19 comprises a heavy chain variable domain and a light chain variable domain, the heavy chain variable domain comprising an amino acid sequence with at least 95% sequence identity to SEQ ID NO: 7, and the light chain variable domain comprising an amino acid sequence with at least 95% sequence identity to SEQ ID NO: 8. In some embodiments, the antigen-binding domain that binds CD19 comprises a heavy chain variable domain and a light chain variable domain, the heavy chain variable domain comprising an amino acid sequence with at least 90% sequence identity to SEQ ID NO: 7, and the light chain variable domain comprising an amino acid sequence with at least 90% sequence identity to SEQ ID NO: 8. In some embodiments, the antigen-binding domain that binds CD19 comprises a heavy chain variable domain and a light chain variable domain, the heavy chain variable domain comprising an amino acid sequence as set forth in SEQ ID NO: 7, and the light chain variable domain comprising an amino acid sequence as set forth in SEQ ID NO: 8.

In some embodiments, the antigen-binding domain that binds CD19 comprises a heavy chain variable domain, the heavy chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 7. In some embodiments, the antigen-binding domain that binds CD19 comprises a heavy chain variable domain, the heavy chain variable domain comprising an amino acid sequence with at least 90% sequence identity to SEQ ID NO: 7. In some embodiments, the antigen-binding domain that binds CD19 comprises a heavy chain variable domain, the heavy chain variable domain comprising an amino acid sequence with at least 95% sequence identity to SEQ ID NO: 7. In some embodiments, the antigen-binding domain that binds CD19 comprises a heavy chain variable domain, the heavy chain variable domain comprising an amino acid sequence as set forth in SEQ ID NO: 7.

In some embodiments, the antigen-binding domain that binds CD19 comprises a light chain variable domain, the light chain variable domain comprising an amino acid sequence with at least 70%, at least 75%, at least 80%, at least 85%, at least 90%, at least 95%, at least 96%, at least 97%, at least 98%, at least 99%, or greater sequence identity to SEQ ID NO: 8. In some embodiments, the antigen-binding domain that binds CD19 comprises a light chain variable domain, the light chain variable domain comprising an amino acid sequence with at least 90% sequence identity to SEQ ID NO: 8. In some embodiments, the antigen-binding domain that binds CD19 comprises a light chain variable domain, the light chain variable domain comprising an amino acid sequence with at least 95% sequence identity to SEQ ID NO: 8. In some embodiments, the antigen-binding domain that binds CD19 comprises a light chain variable domain, the light chain variable domain comprising an amino acid sequence as set forth in SEQ ID NO: 8.

In some embodiments, the antigen-binding domain that binds CD19 comprises a spacer sequence between two domains or components. In some embodiments, an antigen-biding domain comprises a spacer sequence between a heavy chain variable domain and a light chain variable domain. In some embodiments, a spacer comprises a sequence as set forth in SEQ ID NO: 9.

In some embodiments, the antigen-binding domain that binds CD19 comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 17. In some embodiments, the antigen-binding domain that binds CD19 comprises an amino acid sequence having at least 90% sequence identity to SEQ ID NO: 17. In some embodiments, the antigen-binding domain that binds CD19 comprises an amino acid sequence as set forth in SEQ ID NO: 17.

In some embodiments, the antigen-binding domain that binds CD19 is encoded by a nucleic acid sequence having at least 70%, at least 75%, at least 80%, at least 85%, at least 90%, at least 95%, at least 96%, at least 97%, at least 98%, at least 99%, or greater sequence identity to SEQ ID NO: 18. In some embodiments, the antigen-binding domain that binds CD19 is encoded by a nucleic acid sequence having at least 90% sequence identity to SEQ ID NO: 18. In some embodiments, an antigen-binding domain is encoded a nucleic acid sequence as set forth in SEQ ID NO: 18.

Other antigen-binding domains that bind CD19 can also be included in the CAR disclosed herein. Exemplary antigen-binding domains are described in International Application Publication No. WO2017062952 and U.S. Application Publication No. US20220220200.

In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NOs: 35, 36, and 37, respectively, and the light chain variable domain comprising CDR1, CDR2, and CDR3 amino acid sequences as set forth in SEQ ID NOs: 38, 39, and 40, respectively. In some embodiments, the heavy chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 29, and the light chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 30.

In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NOs: 41, 42, and 43, respectively, and the light chain variable domain comprising CDR1, CDR2, and CDR3 amino acid sequences as set forth in SEQ ID NOs: 44, 45, and 46, respectively. In some embodiments, the heavy chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 31, and the light chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 32.

In some embodiments, the antigen-binding domain that binds CD19 comprises a heavy chain variable domain and a light chain variable domain, the heavy chain variable domain comprising CDR1, CDR2, and CDR3 amino acid sequences as set forth in SEQ ID NOs: 47, 48, and 49, respectively, and the light chain variable domain comprising CDR1, CDR2, and CDR3 amino acid sequences as set forth in SEQ ID NOs: 50, 51, and 52, respectively. In some embodiments, the heavy chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 33, and the light chain variable domain comprises an amino acid sequence with at least 70%, at least 75%, at least 80%, at least 85%, at least 90%, at least 95%, at least 96%, at least 97%, at least 98%, at least 99%, or greater sequence identity to SEQ ID NO: 34.

In some embodiments, the extracellular domain of the CAR further comprises a hinge region. In some embodiments, a hinge region is positioned between (e.g., links together), an extracellular domain and a transmembrane domain. In some embodiments, the hinge region is a short sequence of amino acids that can facilitate structural flexibility between polypeptide domains, e.g., between an extracellular domain and a transmembrane domain (see, e.g. Woof et al., Nat. Rev. Immunol. 4(2):89-99 (2004)). In some embodiments, a hinge region may include all, or a portion of, an extracellular region of any suitable transmembrane protein (e.g., CD8α).

In some embodiments, the hinge region is derived from a CD8α protein or a CD28 protein. In some embodiments, a hinge region is derived from a CD8α protein. In some embodiments, the hinge region is derived from a CD28 protein. In some embodiments, a hinge region is or comprises a hinge region or functional fragment thereof from a CD28 protein. In some embodiments, the hinge region is or comprises a hinge region or functional fragment thereof from a CD8α protein. In some embodiments, the hinge region is derived from a human CD8α protein or a human CD28 protein. In some embodiments, the hinge region is derived from a human CD8α protein. In some embodiments, the hinge region is derived from a human CD28 protein. In some embodiments, the hinge region is or comprises a hinge region or functional fragment thereof from a human CD28 protein. In some embodiments, the hinge region is or comprises a hinge region or functional fragment thereof from a human CD8α protein.

In some embodiments, a hinge region comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 28. In some embodiments, a hinge region comprises an amino acid sequence as set forth in SEQ ID NO: 28.

In some embodiments, a hinge region is derived from the same polypeptide as a transmembrane domain. In some embodiments, a hinge region and a transmembrane domain are derived from a CD8 polypeptide. In some embodiments, a hinge region and a transmembrane domain are derived from a CD8α polypeptide. In some embodiments, a hinge region and transmembrane domain comprise an amino acid sequence having at least 70%, at least 75%, at least 80%, at least 85%, at least 90%, at least 95%, at least 96%, at least 97%, at least 98%, at least 99%, or greater sequence identity to SEQ ID NO: 19. In some embodiments, a hinge region and transmembrane domain comprise an amino acid sequence having at least 90% sequence identity to SEQ ID NO: 19. In some embodiments, a hinge region and transmembrane domain comprise an amino acid sequence as set forth in SEQ ID NO: 19.

In some embodiments, a hinge region and transmembrane domain are encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 20. In some embodiments, a hinge region and transmembrane domain are encoded by a nucleic acid sequence having at least 90% sequence identity to SEQ ID NO: 20. In some embodiments, a hinge region and transmembrane domain are encoded by a nucleic acid sequence as set forth in SEQ ID NO: 20.

### Transmembrane Domain

In some embodiments, the transmembrane domain of the CAR is derived from a natural source (e.g., a natural or wild-type polypeptide). In some embodiments, the transmembrane domain, as used in accordance with the present disclosure, is derived from any suitable transmembrane protein or polypeptide known in the art. In some embodiments, a transmembrane domain is derived from a CD3 epsilon polypeptide, a CD4 polypeptide, a CD5 polypeptide, a CD8 polypeptide, a CD9 polypeptide, a CD16 polypeptide, a CD22 polypeptide, a CD28 polypeptide, a CD33 polypeptide, a CD37 polypeptide, a CD45 polypeptide, a CD64 polypeptide, a CD80 polypeptide, a CD86 polypeptide, a CD134 polypeptide, a CD137 polypeptide, a CD154 polypeptide, a T cell receptor alpha chain polypeptide, a T cell receptor beta chain polypeptide, a T cell receptor zeta chain polypeptide, or any combination thereof. In some embodiments, a transmembrane is or comprises a transmembrane domain or functional fragment thereof from a CD3 epsilon polypeptide, a CD4 polypeptide, a CD5 polypeptide, a CD8 polypeptide, a CD9 polypeptide, a CD16 polypeptide, a CD22 polypeptide, a CD28 polypeptide, a CD33 polypeptide, a CD37 polypeptide, a CD45 polypeptide, a CD64 polypeptide, a CD80 polypeptide, a CD86 polypeptide, a CD134 polypeptide, a CD137 polypeptide, a CD154 polypeptide, a T cell receptor alpha chain polypeptide, a T cell receptor beta chain polypeptide, a T cell receptor zeta chain polypeptide, or any derivatives thereof and/or any combination thereof. In some embodiments, a transmembrane is synthetically derived, or engineered. In some embodiments, a synthetically derived or engineered transmembrane domain comprises predominantly hydrophobic residues (e.g., leucine, valine, etc.). In some embodiments, an engineered transmembrane domain is or comprises any engineered transmembrane domain known in the field.

The present disclosure appreciates that CD8 is a transmembrane glycoprotein that functions as a co-receptor for the T-cell receptor (TCR), and is expressed primarily on the surface of T-cells (e.g., cytotoxic T-cells). The most common form of CD8 exists as a dimer composed of a CD8α and CD8β chain. In some embodiments, a transmembrane domain is derived from a CD8α protein. In some embodiments, a transmembrane protein comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 11. In some embodiments, a transmembrane protein comprises an amino acid sequence as set forth in SEQ ID NO: 11.

The present disclosure further appreciates that CD28 is expressed on T-cells and provides co-stimulatory signals required for T-cell activation. CD28 is the receptor for CD80 (B7.1) and CD86 (B7.2). In some embodiments, a CAR of the present disclosure comprises a CD28 transmembrane domain. In some embodiments, the transmembrane protein comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 12. In some embodiments, a transmembrane protein comprises an amino acid sequence as set forth in SEQ ID NO: 12.

### Intracellular Signaling Domain

In some embodiments, an intracellular signaling domain of the CAR disclosed herein is derived from a polypeptide found in humans (e.g., an intracellular signaling domain or fragment thereof found in any suitable human polypeptide). In some embodiments, the intracellular signaling domain provided herein is derived from a 4-1BB polypeptide, a B7-H3 polypeptide, a CD2 polypeptide, a CD3 gamma polypeptide, a CD3 delta polypeptide, a CD3 zeta polypeptide, a CD7 polypeptide, a CD27 polypeptide, a CD28 polypeptide, a CD30 polypeptide, a CD40 polypeptide, an FcεRI polypeptide (e.g., an FcεRI gamma chain polypeptide), an FcγRI polypeptide, LIGHT polypeptide, NKG2C polypeptide, OX40 polypeptide, PD-1 polypeptide, or any derivatives thereof or any combination thereof. In some embodiments, the intracellular signaling domain is derived from a CD3 zeta polypeptide. In some embodiments, the intracellular signaling domain is derived from a CD28 polypeptide. In some embodiments, the intracellular signaling domain is derived from a CD28 polypeptide and a CD3 zeta polypeptide.

In some embodiments, the intracellular signaling domain comprises at least one intracellular signaling domain or functional fragment thereof from a 4-1BB polypeptide, a B7-H3 polypeptide, a CD2 polypeptide, a CD3 gamma polypeptide, a CD3 delta polypeptide, a CD3 zeta polypeptide, a CD7 polypeptide, a CD27 polypeptide, a CD28 polypeptide, a CD30 polypeptide, a CD40 polypeptide, an FcεRI polypeptide (e.g., an FcεRI gamma chain polypeptide), an FcγRI polypeptide, LIGHT polypeptide, NKG2C polypeptide, OX40 polypeptide, PD-1 polypeptide, or any derivatives thereof or any combination thereof. In some embodiments, the intracellular signaling domain comprises an intracellular signaling domain or functional fragment thereof from a CD3 zeta polypeptide. In some embodiments, the intracellular signaling domain comprises an intracellular signaling domain or functional fragment thereof from a CD28 polypeptide. In some embodiments, the intracellular signaling domain comprises an intracellular signaling domain or functional fragment thereof from a CD28 polypeptide and an intracellular signaling domain or functional fragment thereof from a CD3 zeta polypeptide. In some embodiments, the intracellular signaling domain comprises, from N-terminus to C-terminus, an intracellular signaling domain or functional fragment thereof from a CD28 polypeptide and an intracellular signaling domain or functional fragment thereof from a CD3 zeta polypeptide.

In some embodiments, the intracellular signaling domain of the present disclosure comprises at least one signaling sequence from a 4-1BB polypeptide, a B7-H3 polypeptide, a CD2 polypeptide, a CD3 gamma polypeptide, a CD3 delta polypeptide, a CD3 zeta polypeptide, a CD7 polypeptide, a CD27 polypeptide, a CD28 polypeptide, a CD30 polypeptide, a CD40 polypeptide, an FcεRI polypeptide (e.g., an FcεRI gamma chain polypeptide), an FcγRI polypeptide, LIGHT polypeptide, NKG2C polypeptide, OX40 polypeptide, PD-1 polypeptide, or any combination thereof. In some embodiments, the intracellular signaling domain comprises at least one signaling sequence from a CD3 zeta polypeptide. In some embodiments, the intracellular signaling domain comprises at least one signaling sequence from a CD28 polypeptide. In some embodiments, the intracellular signaling domain comprises at least one signaling sequence from a CD28 polypeptide and at least one signaling sequence from a CD3 zeta polypeptide. In some embodiments, the intracellular signaling domain comprises, from N-terminus to C-terminus, at least one signaling sequence from a CD28 polypeptide and at least one signaling sequence from a CD3 zeta polypeptide.

In some embodiments, an intracellular signaling domain is or comprises at least one signaling sequence or signaling motif. In some embodiments, a signaling sequence (or signaling motif) comprises one or more (e.g., two, three, four, five, or more) co-stimulatory domains (e.g., any co-stimulatory domain described herein). In some embodiments, a signaling sequence comprises one co-stimulatory domain. In some embodiments, a signaling sequence comprises two co-stimulatory domains. In some embodiments, a signaling sequence comprises three co-stimulatory domains. In some embodiments, a signaling sequence comprises two or more of the same co-stimulatory domains. In some embodiments, a signaling sequence comprises two or more different co-stimulatory domains.

In some embodiments, a signaling sequence as used in accordance with the present disclosure is or comprises one or more immunoreceptor tyrosine-based activation motifs (ITAMs). In some embodiments, a signal sequence is or comprises a consensus sequence of YXXL/I, where Y is a tyrosine residue, L/I is a leucine or isoleucine residue, and X is any amino acid residue. In some embodiments, a signal sequence is or comprises a consensus sequences of YXXL/IX₍₆₋₈₎YXXL, where Y is a tyrosine residue, L/I is a leucine or isoleucine residue, and X is any amino acid residue. In some embodiments, a signaling sequence comprises a YNMN (SEQ ID NO: 53) motif. In some embodiments, a signaling sequence comprises at least one ITAM sequence from a CD3 polypeptide (e.g., a CD3 zeta polypeptide). In some embodiments, a signaling sequence comprises at least one ITAM sequence from a CD28 polypeptide.

It is understood that the most common intracellular signaling domain used in CAR therapies is an intracellular signaling domain of CD3 zeta (CD3ζ). CD3 zeta associates with T cell receptors to produce a signal and contains ITAMs. In some embodiments, an intracellular signaling domain is or comprises a CD3 zeta intracellular signaling domain. In some embodiments, an intracellular signaling domain comprises an intracellular signaling domain or a functional fragment thereof from a CD3 zeta polypeptide.

In some embodiments, an intracellular signaling domain comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 23. In some embodiments, an intracellular signaling domain comprises an amino acid sequence as set forth in SEQ ID NO: 23.

In some embodiments, an intracellular signaling domain is encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 24. In some embodiments, an intracellular signaling domain is encoded by a nucleic acid sequence as set forth in SEQ ID NO: 24.

In some embodiments, an intracellular signaling domain comprises a CD28 intracellular signaling domain. In some embodiments, an intracellular signaling domain comprises an intracellular signaling domain or a functional fragment thereof from a CD28 polypeptide. In some embodiments, a CD28 polypeptide intracellular signaling domain or functional fragment thereof comprises a co-stimulatory domain.

In some embodiments, an intracellular signaling domain disclosed herein comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 21. In some embodiments, the intracellular signaling domain comprises an amino acid sequence as set forth in SEQ ID NO: 21.

In some embodiments, an intracellular signaling domain is encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 22. In some embodiments, an intracellular signaling domain is encoded by a nucleic acid sequence as set forth in SEQ ID NO: 22.

### Chimeric Antigen Receptors (CARs)

In some embodiments, a CAR of the present disclosure comprises an extracellular domain, a hinge domain, a transmembrane domain, and an intracellular signaling domain. In some embodiments, a CAR of the present disclosure comprises a signal peptide sequence (also referred to as a targeting signal, localization signal, localization sequence, leader sequence, or leader peptide), an extracellular domain, a hinge domain, a transmembrane domain, and an intracellular signaling domain. In some embodiments, a CAR of the present disclosure comprises, from N-terminus to C-terminus, an extracellular domain, a hinge domain, a transmembrane domain, and an intracellular signaling domain. In some embodiments, a CAR of the present disclosure comprises, from N-terminus to C-terminus, a signal peptide sequence, an extracellular domain, a hinge domain, a transmembrane domain, and an intracellular signaling domain. In some embodiments, the signal peptide sequence is cleaved from the CAR during or after its insertion into a membrane (e.g., ER membrane) during synthesis of the CAR protein. In some embodiments, domains or components (e.g., extracellular domains, hinge regions, transmembrane domains, intracellular signaling domains, etc.) of a CAR are directly linked, or are contiguous. In some embodiments, domains or components of a CAR are not-directly linked, or are non-contiguous.

In some embodiments, a CAR as described herein comprises an intracellular signaling domain, wherein the intracellular signaling domain comprises: (a) a CD3 zeta intracellular signaling domain or functional fragment thereof; and (b) at least one of a 4-1BB, an OX40, or a CD28 intracellular signaling domain or functional fragment thereof. In some embodiments, a 4-1BB intracellular signaling domain or functional fragment thereof, an OX40 intracellular signaling domain, and/or a CD28 intracellular signaling domain or functional fragment thereof is or comprises a co-stimulatory domain.

In some embodiments, a CAR of the present disclosure comprises: (a) a CD28 transmembrane domain; and (b) an intracellular signaling domain comprising: (i) a CD3ζ intracellular signaling domain or functional fragment thereof; and (ii) a CD28 intracellular signaling domain or functional fragment thereof. In some embodiments, a CD28 intracellular signaling domain or functional fragment thereof is or comprises a CD28 co-stimulatory domain.

In some embodiments, a CAR of the present disclosure comprises: (a) a CD8α transmembrane domain; (b) an intracellular signaling domain comprising: (i) a CD3ζ intracellular signaling domain or functional fragment thereof; and (ii) a CD28, an FcεRI gamma chain, and/or a 4-1BB intracellular signaling domain or functional fragment thereof. In some embodiments, a CAR of the present disclosure comprises: (a) a CD8α transmembrane domain; (b) an intracellular signaling domain comprising: (i) a CD3ζ intracellular signaling domain or functional fragment thereof, and (ii) a CD28, an FcεRI gamma chain, and a 4-1BB intracellular signaling domain or functional fragment thereof. In some embodiments, a CAR of the present disclosure comprises: (a) a CD8α transmembrane domain; (b) an intracellular signaling domain comprising: (i) a CD3ζ intracellular signaling domain or functional fragment thereof; and (ii) an FcεRI gamma chain intracellular signaling domain or functional fragment thereof. In some embodiments, a CAR of the present disclosure comprises: (a) a CD8α transmembrane domain; (b) an intracellular signaling domain comprising: (i) a CD3ζ intracellular signaling domain or functional fragment thereof; and (ii) a 4-1BB intracellular signaling domain or functional fragment thereof. In some embodiments, a CD28 intracellular signaling domain or functional fragment thereof is or comprises a CD28 co-stimulatory domain. In some embodiments, a FcεRI intracellular signaling domain or functional fragment thereof is or comprises a FcεRI co-stimulatory domain. In some embodiments, a 4-1BB intracellular signaling domain or functional fragment thereof is or comprises a 4-1BB co-stimulatory domain.

In some embodiments, a CAR of the present disclosure comprises (a) a CD8α transmembrane domain, and (b) an intracellular signaling domain comprising: (i) a CD3ζ intracellular signaling domain or functional fragment thereof, and (ii) a CD27 and/or a CD28 intracellular signaling domain or functional fragment thereof. In some embodiments, a CD27 intracellular signaling domain or functional fragment thereof is or comprises a CD27 co-stimulatory domain. In some embodiments, a CD28 intracellular signaling domain or functional fragment thereof is or comprises a CD28 co-stimulatory domain.

In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a CD27, a 4-1BB, and/or an FcεRI gamma chain intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a CD27, a 4-1BB, and an FcεRI gamma chain intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a CD27 intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof; and (ii) a 4-1BB intracellular signaling domain or functional fragment thereof. In some embodiments, a CAR of the present disclosure comprises (a) a CD28 transmembrane domain, and (b) an intracellular signaling domain comprising: (i) a CD3ζ intracellular signaling domain or functional fragment thereof, and (ii) an FcεRI gamma chain intracellular signaling domain or functional fragment thereof. In some embodiments, a CD27 intracellular signaling domain or functional fragment thereof is or comprises a CD27 co-stimulatory domain. In some embodiments, a FcεRI intracellular signaling domain or functional fragment thereof is or comprises a FcεRI co-stimulatory domain. In some embodiments, a 4-1BB intracellular signaling domain or functional fragment thereof is or comprises a 4-1BB co-stimulatory domain.

The present disclosure also includes functional variants of any of CAR, or CAR domain/component, described herein. CAR functional variants encompass, for example, variants of a CAR described herein (a parent CAR) that retains the ability to recognize a particular target cell to a similar extent, the same extent, or to a higher extent, as the parent CAR. In reference to a nucleic acid sequence encoding a parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99% identical to the nucleic acid sequence encoding the parent CAR. In some embodiments, a parent CAR comprises an amino acid sequence as set forth in SEQ ID NO: 10 or 13. Alternatively or additionally, in some embodiments, a CAR functional variant comprises the amino acid sequence of a parent CAR with at least one non-conservative amino acid substitution. In some embodiments, a non-conservative amino acid substitution does not compromise or inhibit a biological activity of a CAR functional variant. In some embodiments, a non-conservative amino acid substitution may enhance a biological activity of a CAR functional variant, such that biological activity of the functional variant is increased relative to its parent CAR.

The present disclosure further provides for CARs comprising an extracellular domain directed to any target molecule of interest (e.g., comprising any of known antigen-binding domain, e.g., antibody, scFv, etc.), and further comprising any transmembrane domain described herein (including any hinge domain described herein), any intracellular signaling domain described herein (including any signal sequences or motifs, any co-stimulatory domains, etc., described herein), present in any combination.

In some embodiments, a CAR comprises: (a) a hinge region, (b) a transmembrane domain derived from a human CD8α polypeptide, (c) an intracellular signaling domain comprising: (i) a human CD3ζ intracellular signaling domain or fragment thereof, and (ii) a human CD28 intracellular signaling domain or fragment thereof, wherein the CD28 intracellular signaling domain or fragment thereof is or comprises a co-stimulatory domain. In some embodiments, a CAR comprises: (a) a hinge region derived from a human CD8α polypeptide, (b) a transmembrane domain derived from a human CD8α polypeptide, (c) an intracellular signaling domain comprising: (i) a human CD3ζ intracellular signaling domain; and (ii) a human CD28 intracellular signaling domain. In some embodiments, a CAR comprises a sequence as set forth in SEQ ID NO: 27.

In some embodiments, a CAR comprises: (a) a hinge region, (b) a transmembrane domain derived from a human CD8α polypeptide, (c) an intracellular signaling domain comprising: (i) a human CD3ζ intracellular signaling domain or fragment thereof, and (ii) a CD27 and/or a CD28 intracellular signaling domain or fragment thereof, wherein the CD27 and/or CD28 intracellular signaling domain or fragment thereof is or comprises a co-stimulatory domain.

In some embodiments, a CAR comprises: (a) a hinge region, (b) a transmembrane domain derived from a human CD8α polypeptide, (c) an intracellular signaling domain comprising: (i) a human CD3ζ intracellular signaling domain or fragment thereof, and (ii) a human CD28, a human CD27, and/or an FcεRI gamma chain intracellular signaling domain or fragment thereof, wherein the human CD28, the human CD27, and/or the FcεRI gamma chain intracellular signaling domain or fragment thereof are or comprise a co-stimulatory domain.

In some embodiments, a CAR can comprises: (a) a hinge region, (b) a transmembrane domain derived from a human CD8α polypeptide, (c) an intracellular signaling domain comprising: (i) a human CD3ζ intracellular signaling domain; and (ii) a human CD28 and/or an FcεRI gamma chain intracellular signaling domain, wherein the CD28 and/or the FcεRI gamma chain intracellular signaling domain or fragment thereof are or comprise a co-stimulatory domain.

In some embodiments, a CAR as described herein, further comprises a signal peptide sequence. In some embodiments, a signal peptide is positioned at the amino terminus of an extracellular domain (e.g., at the N-terminus of an antigen-binding domain). A signal peptide as used in accordance with the present disclosure may comprise any suitable signal peptide sequence. In some embodiments, a signal peptide sequence is a human granulocyte macrophage colony-stimulating factor (GM-CSF) receptor signal peptide sequence or a CD8α signal peptide sequence. In some embodiments, a CAR provided herein comprises a human scFv comprising a CD8α signal peptide sequence. In some embodiments, a signal peptide sequence comprises an amino acid sequence as set forth in SEQ ID NO: 15.

In some embodiments, a provided CAR comprises: (a) a CD8α hinge region comprising SEQ ID NO: 28, (b) a CD8α transmembrane domain comprising SEQ ID NO: 11, (c) a CD28 intracellular signaling domain comprising SEQ ID NO: 21, and (d) a CD3ζ intracellular signaling domain comprising SEQ ID NO: 23. In some embodiments, a provided CAR comprises, from N-terminus to C-terminus: (a) a CD8α hinge region comprising SEQ ID NO: 28, (b) a CD8α transmembrane domain comprising SEQ ID NO: 11, (c) a CD28 intracellular signaling domain comprising SEQ ID NO: 21, and (d) a CD3ζ intracellular signaling domain comprising SEQ ID NO: 23.

In some embodiments, a provided CAR comprises: (a) an antigen-binding domain comprising SEQ ID NO: 17, (b) a CD8α hinge region comprising SEQ ID NO: 28, (c) a CD8α transmembrane domain comprising SEQ ID NO: 11, (d) a CD28 intracellular signaling domain comprising SEQ ID NO: 21, and (e) a CD3ζ intracellular signaling domain comprising SEQ ID NO: 23. In some embodiments, a provided CAR comprises, from N-terminus to C-terminus: (a) an antigen-binding domain comprising SEQ ID NO: 17, (b) a CD8α hinge region comprising SEQ ID NO: 28, (c) a CD8α transmembrane domain comprising SEQ ID NO: 11, (d) a CD28 intracellular signaling domain comprising SEQ ID NO: 21, and (e) a CD3ζ intracellular signaling domain comprising SEQ ID NO: 23.

In some embodiments, a provided CAR comprises: (a) a CD8α signal peptide sequence comprising SEQ ID NO: 15, (b) an antigen-binding domain comprising SEQ ID NO: 17, (c) a CD8α hinge region as set forth in SEQ ID NO: 28, (d) a CD8α transmembrane domain as set forth in SEQ ID NO: 11, (e) a CD28 intracellular signaling domain as set forth in SEQ ID NO: 21, and (f) a CD3ζ intracellular signaling domain as set forth in SEQ ID NO: 23. In some embodiments, a provided CAR comprises, from N-terminus to C-terminus: (a) a CD8α signal peptide sequence comprising SEQ ID NO: 15, (b) an antigen-binding domain comprising SEQ ID NO: 17, (c) a CD8α hinge region as set forth in SEQ ID NO: 28, (d) a CD8α transmembrane domain as set forth in SEQ ID NO: 11, (e) a CD28 intracellular signaling domain as set forth in SEQ ID NO: 21, and (f) a CD3ζ intracellular signaling domain as set forth in SEQ ID NO: 23.

In some embodiments, a CAR of the present disclosure comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 10. In some embodiments, a CAR of the present disclosure comprises an amino acid sequence having at least 80% sequence identity to SEQ ID NO: 10. In some embodiments, a CAR of the present disclosure comprises an amino acid sequence having at least 85% sequence identity to SEQ ID NO: 10. In some embodiments, a CAR of the present disclosure comprises an amino acid sequence having at least 90% sequence identity to SEQ ID NO: 10. In some embodiments, a CAR of the present disclosure comprises an amino acid sequence having at least 95% sequence identity to SEQ ID NO: 10. In some embodiments, a CAR of the present disclosure comprises an amino acid sequence having at least 96% sequence identity to SEQ ID NO: 10. In some embodiments, a CAR of the present disclosure comprises an amino acid sequence having at least 97% sequence identity to SEQ ID NO: 10. In some embodiments, a CAR of the present disclosure comprises an amino acid sequence having at least 98% sequence identity to SEQ ID NO: 10. In some embodiments, a CAR of the present disclosure comprises an amino acid sequence having at least 99% sequence identity to SEQ ID NO: 10. In some embodiments, a CAR of the present disclosure comprises an amino acid sequence as set forth in SEQ ID NO: 10.

In some embodiments, a CAR of the present disclosure comprises an amino acid sequence having at least 70%, at least 75%, at least 80%, at least 85%, at least 90%, at least 95%, at least 96%, at least 97%, at least 98%, at least 99%, or greater sequence identity to SEQ ID NO: 13. In some embodiments, a CAR of the present disclosure comprises an amino acid sequence having at least 80% sequence identity to SEQ ID NO: 13. In some embodiments, a CAR of the present disclosure comprises an amino acid sequence having at least 85% sequence identity to SEQ ID NO: 13. In some embodiments, a CAR of the present disclosure comprises an amino acid sequence having at least 90% sequence identity to SEQ ID NO: 13. In some embodiments, a CAR of the present disclosure comprises an amino acid sequence having at least 95% sequence identity to SEQ ID NO: 13. In some embodiments, a CAR of the present disclosure comprises an amino acid sequence having at least 96% sequence identity to SEQ ID NO: 13. In some embodiments, a CAR of the present disclosure comprises an amino acid sequence having at least 97% sequence identity to SEQ ID NO: 13. In some embodiments, a CAR of the present disclosure comprises an amino acid sequence having at least 98% sequence identity to SEQ ID NO: 13. In some embodiments, a CAR of the present disclosure comprises an amino acid sequence having at least 99% sequence identity to SEQ ID NO: 13. In some embodiments, a CAR of the present disclosure comprises an amino acid sequence as set forth in SEQ ID NO: 13.

In some embodiments, a CAR of the present disclosure is encoded by nucleic acid sequence having at least 70%, at least 75%, at least 80%, at least 85%, at least 90%, at least 95%, at least 96%, at least 97%, at least 98%, at least 99%, or greater sequence identity to SEQ ID NO: 14. In some embodiments, a CAR of the present disclosure is encoded by a nucleic acid sequence having at least 80% sequence identity to SEQ ID NO: 14. In some embodiments, a CAR of the present disclosure is encoded by a nucleic acid sequence having at least 85% sequence identity to SEQ ID NO: 14. In some embodiments, a CAR of the present disclosure is encoded by a nucleic acid sequence having at least 90% sequence identity to SEQ ID NO: 14. In some embodiments, a CAR of the present disclosure is encoded by a nucleic acid sequence having at least 95% sequence identity to SEQ ID NO: 14. In some embodiments, a CAR of the present disclosure is encoded by a nucleic acid sequence having at least 96% sequence identity to SEQ ID NO: 14. In some embodiments, a CAR of the present disclosure is encoded by a nucleic acid sequence having at least 97% sequence identity to SEQ ID NO: 14. In some embodiments, a CAR of the present disclosure is encoded by a nucleic acid sequence having at least 98% sequence identity to SEQ ID NO: 14. In some embodiments, a CAR of the present disclosure is encoded by a nucleic acid sequence having at least 99% sequence identity to SEQ ID NO: 14. In some embodiments, a CAR of the present disclosure is encoded by a nucleic acid sequence as set forth in SEQ ID NO: 14.

It has been observed that T cells engineered to express a CD19-CAR that incorporates a 4-1BB costimulatory domain produced substantially higher background levels of IFNγ, in the absence CD19-expressing target cells, than T cells engineered to express a CD19-CAR that incorporates a costimulatory domain derived from CD28, CD27, or FcεRI gamma chain. Without wishing to be bound by theory, it is hypothesized that the higher background level of cytokine production is undesirable for treating autoimmune diseases. Therefore, in certain embodiments, a CAR disclosed herein does not comprise an intracellular T cell signaling domain derived from 4-1BB.

### Nucleic Acid Constructs

The present disclosure further provides for an engineered nucleic acid, or nucleic acid construct, comprising a nucleic acid sequence that encodes any polypeptide described herein, e.g., any CAR described herein. In some embodiments, an engineered nucleic acid comprises a promoter operably linked to a nucleic acid sequence that encodes a CAR (e.g., any CAR described herein). Any appropriate promoter may be operably linked to any of the engineered nucleic acid sequences described herein. Non-limiting examples of promoters that may be used in accordance with the present disclosure include EF1a, SFFV, PGK, CMV, CAG, UbC, murine stem cell virus (MSCV), MND, EF1a hybrid promoters, CAG hybrid promoters, or derivatives or functional fragments thereof. In some embodiments, a promoter is an EF1a promoter. In some embodiments, promoter is a SFFV promoter. In some embodiments, a promoter is a PGK promoter. In some embodiments, a promoter is a CMV promoter. In some embodiments, a promoter is a CAG promoter. In some embodiments, a promoter is a UbC promoter. In some embodiments, a promoter is a MSCV promoter. In some embodiments, a promoter is a MND promoter.

In some cases, an engineered nucleic acid comprises sufficient cis-acting elements (e.g., a promoter and/or an enhancer) that supplement expression of a provided engineered nucleic acid sequence where the remaining elements needed for expression can be supplied by a host cell (e.g., a mammalian cell, e.g., a T cell) or in an in vitro expression system.

The present disclosure also provides for vectors, or plasmids, comprising any engineered nucleic acid as described herein. The present disclosure provides for transposons, cosmids, viral vectors (e.g., any adenoviral vectors (e.g., pSV or pCMV vectors), adeno-associated virus (AAV) vectors, lentivirus vectors, and retroviral vectors), and any Gateway® vectors comprising any engineered nucleic acid described herein. In some embodiments, a viral vector is selected from the group consisting of: a lentiviral vector, a retroviral vector, an adenoviral vector, and an adeno-associated viral (AAV) vector. In some embodiments, a viral vector is a lentiviral vector. In some embodiments, a viral vector is a retroviral vector. In some embodiments, a viral vector is an adenoviral vector. In some embodiments, a viral vector is a AAV vector.

Exemplary lentiviral vectors that may be used in accordance with the present disclosure include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by a gene of interest or expression cassette of interest (e.g., an engineered nucleic acid as described here). Most often, the structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal. Accordingly, in some embodiments, a minimum retroviral vector comprises from 5′ to 3′: a 5′ long terminal repeat (LTR), a packaging signal, an optional exogenous promoter and/or enhancer, an exogenous gene of interest (or engineered nucleic acid), and a 3′ LTR. In some embodiments, if no exogenous promoter is provided, gene expression may be driven by the 5′ LTR, which is a weak promoter and requires the presence of Tat to activate expression. In many embodiments, structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

Nucleic acids (e.g., genes) to be packaged into a retrovirus (e.g., a lentivirus) can be incorporated into the proviral backbone in several general ways. The most straightforward constructions are ones in which the structural genes of the retrovirus are replaced by a single gene which then is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, nucleic acids (e.g., genes) to be packaged into a retrovirus are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In some embodiments, a retroviral vector comprises a modified 5′ LTR and/or 3′ LTR. Modifications of the 3′ LTR are often made to improve the safety of lentiviral or retroviral systems by rendering viruses replication-defective. In some embodiments, a retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In some embodiments, a 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in some embodiments of the present disclosure.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent to a 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. Virology, 69(4):2101-09). The packaging signal may be a minimal packaging signal (also referred to as the psi [Ψ] sequence) needed for encapsidation of the viral genome.

In some embodiments, a retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) Cell 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In some embodiments, retroviral vector backbones comprise one or more FLAP elements upstream or downstream of the heterologous genes of interest in the vectors. For example, in some embodiments, a transfer plasmid includes a FLAP element. In some embodiments, a vector of the present disclosure comprises a FLAP element isolated from HIV-1.

In some embodiments, a retroviral vector (e.g., lentiviral vector) further comprises an export element. In some embodiments, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. Virol. 65: 1053; and Cullen et al., (1991) Cell 58: 423) and the hepatitis B virus post-transcriptional regulatory element (HPRE). Generally, the RNA export element is placed within the 3′ UTR of a gene, and can be inserted as one or multiple copies.

In some embodiments, a retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. Virol., 73:2886); the posttranscriptional regulatory element present in hepatitis B virus (HPRE) (Huang et al., Mol. Cell. Biol., 5:3864); an optimized posttranscriptional regulatory element (oPRE; see Schambach et al., (2006) Gene Therapy 13, 641-45); and the like (Liu et al., (1995), Genes Dev., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In some embodiments, vectors of the present disclosure lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors of the present disclosure lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in some embodiments, a retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the present disclosure, include an ideal polyadenylation sequence (e.g., AATAAA, ATTAAA AGTAAA), a bovine growth hormone polyadenylation sequence (BGHpA), a rabbit β-globin polyadenylation sequence (rβgpA), or another suitable heterologous or endogenous polyadenylation sequence known in the art.

In some embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) Proc. Natl. Acad. Sci., USA, 99:16433; and Zhan et al., 2001, Hum. Genet., 109:471). In some embodiments, a retroviral vector comprises an insulator element in one or both LTRs or elsewhere in the region of the vector that integrates into the cellular genome. Suitable insulators for use in the present disclosure include, but are not limited to, the chicken β-globin insulator (see Chung et al., (1993). Cell 74:505; Chung et al., (1997) Proc. Natl. Acad. Sci., USA 94:575; and Bell et al., 1999. Cell 98:387). Examples of insulator elements include, but are not limited to, an insulator from a β-globin locus, such as chicken HS4.

Non-limiting examples of lentiviral vectors include pLVX-EF1alpha-AcGFP1-C1 (Clontech Catalog #631984), pLVX-EF1alpha-IRES-mCherry (Clontech Catalog #631987), pLVX-Puro (Clontech Catalog #632159), pLVX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

In some embodiments, an engineered nucleic acid of the present disclosure comprises a nucleic acid sequence encoding a CAR, wherein the nucleic acid sequence has at least 70%, at least 75%, at least 80%, at least 85%, at least 90%, at least 95%, at least 96%, at least 97%, at least 98%, at least 99%, or greater sequence identity to SEQ ID NO: 14. In some embodiments, an engineered nucleic acid of the present disclosure comprises a nucleic acid sequence having at least 80% sequence identity to SEQ ID NO: 14. In some embodiments, an engineered nucleic acid of the present disclosure comprises a nucleic acid sequence having at least 85% sequence identity to SEQ ID NO: 14. In some embodiments, an engineered nucleic acid of the present disclosure comprises a nucleic acid sequence having at least 90% sequence identity to SEQ ID NO: 14. In some embodiments, an engineered nucleic acid of the present disclosure comprises a nucleic acid sequence having at least 95% sequence identity to SEQ ID NO: 14. In some embodiments, an engineered nucleic acid of the present disclosure comprises a nucleic acid sequence having at least 96% sequence identity to SEQ ID NO: 14. In some embodiments, an engineered nucleic acid of the present disclosure comprises a nucleic acid sequence having at least 97% sequence identity to SEQ ID NO: 14. In some embodiments, an engineered nucleic acid of the present disclosure comprises a nucleic acid sequence having at least 98% sequence identity to SEQ ID NO: 14. In some embodiments, an engineered nucleic acid of the present disclosure comprises a nucleic acid sequence having at least 99% sequence identity to SEQ ID NO: 14. In some embodiments, an engineered nucleic acid of the present disclosure comprises a nucleic acid sequence as set forth in SEQ ID NO: 14.

In certain embodiments, a lentivirus vector disclosed herein comprises a truncated 5′ LTR (e.g., with deletion of its U3 region), an HIV-1Ψ packaging sequence, a MSCV promoter operably linked to a nucleic acid encoding a CAR (e.g., any of the CARs as disclosed herein), and a truncated 3′ LTR (e.g., with deletion of its U3 region). In certain embodiments, the lentivirus vector further comprises a RRE, a cPPT/CTS, and/or an oPRE. In certain embodiments, the lentivirus vector comprises a truncated 5′ LTR (e.g., with deletion of its U3 region), an HIV-1Ψ packaging sequence, a RRE, a cPPT/CTS, a MSCV promoter operably linked to a nucleic acid encoding a CAR (e.g., any of the CARs as disclosed herein), an oPRE, and a truncated 3′ LTR (e.g., with deletion of its U3 region). In certain embodiments, the lentivirus vector is pseudotyped with VSV-G envelope protein.

### Methods of Making Engineered T Cells

Also provided herein are methods of making an engineered T cell, the methods comprising introducing into a host T cell an engineered nucleic acid comprising a nucleic acid sequence encoding a CAR (e.g., any CAR described herein). Accordingly in some embodiments, an engineered T cell refers to a genetically modified T cell that has been modified to express a CAR, e.g., any provided anti-CD19 CAR.

In some embodiments, a host T cell used to make an engineered T cell can be any T cell such as a cultured T cell, e.g., a primary T cell, or a T cell derived from a cultured T cell line, e.g., a Jurkat, SupT1, etc., or a T cell obtained from a mammal. In some embodiments, a T cell used to make an engineered T cell can be selected from naive T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. In some embodiments, a host T cell used to make an engineered T cell can be a CD3+ cell. In some embodiments, a host T cell can be CD4+, CD8+, or CD4+ and CD8+. In some embodiments, a host T cell can be any type of T cell, e.g., CD4+/CD8+ double positive T cells, CD4+ helper T cells (e.g., Th1 and Th2 cells), CD8+ T cells (e.g., cytotoxic T cells), memory T cells, naive T cells, regulatory T cells, γδT cells, etc. In some embodiments, a host T cell used to make an engineered T cell can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). In some embodiments, obtained host T cells are substantially free of non-T cells.

The T cells can be obtained from various biological samples of a subject (e.g., a human subject). Non-limiting examples of biological sample include cells, tissue (e.g., tissue obtained by biopsy), blood, serum, plasma, or any sample derived therefrom. In certain embodiments, the sample is a whole blood sample or an apheresis (e.g., leukapheresis) sample obtained from the subject. In certain embodiments, the method comprises obtaining the sample from the subject. In certain embodiments, the method comprises having obtained the sample from the subject.

In certain embodiments, the T cells are isolated from the sample. Isolation of T cells may include an initial purification of T cells from a mixture of plasma, lymphocytes, platelets, red blood cells, monocytes, and granulocytes. Methods for isolation of T cells from a biological sample, such as a whole blood sample or a leukapheresis sample, are well-known. Exemplary methods may include leukapheresis, elutriation, density gradient centrifugation, enrichment by selection, and the like. For example, the method may include obtaining or having obtained a biological sample, such as a fresh, refrigerated, frozen, or cryopreserved leukapheresis product or alternative source of hematopoietic tissue, such as a whole blood sample, bone marrow sample, or a tumor or organ biopsy or removal (e.g., thymectomy) from an entity, such as a laboratory, hospital, or healthcare provider, and performing the aforementioned isolation steps to produce an enriched population of T cells (e.g., starting population of T cells) suitable for expression of a heterologous protein.

Furthermore, the purity of the T cell population can be increased by using one or more selection steps, such as negative selection or positive selection. Negative selection typically involves removal of undesired cell types from a mixed population of cells in a sample using one or more agents that selectively bind to the undesired cell type, whereas positive selection typically involves isolation of the desired cell population using one or more agents that selectively bind to the desired cell type. Enrichment of a T cell population by negative selection can be accomplished, for example, with a combination of antibodies directed to surface markers unique to the negatively selected cells. One method is cell sorting and/or selection via negative magnetic immuno-adherence or flow cytometry that uses a cocktail of monoclonal antibodies directed to cell surface markers present on the negatively selected cells. For example, to enrich for CD4+ cells by negative selection, a monoclonal antibody cocktail can include antibodies to CD14, CD20, CDb, CD16, HLA-DR, and CD8. On the other hand, a positive selection step can be used to specifically select for the desired cell type. Positive selection of T cells can, in certain embodiments, include incubation of a mixed population of cells that contains the T cells with a CD3-binding agent (e.g., anti-CD3 antibody-conjugated beads) for a time sufficient for positive selection of the desired T cells.

In some embodiments, engineered T cells are made using a mixture of cells (e.g., a mixture of host cells). For example, a mixture of cells may be obtained (e.g., from a subject), and an engineered nucleic acid may be inserted into the mixture of cells such that a mixture of engineered cells is made. In some embodiments, a mixture of cells comprises a mixture of T cells (e.g., any T cells described herein). In some embodiments, a mixture of cells comprises CD4+ and/or CD8+ T cells. In some embodiments, a mixture of cells comprises CD4+ and CD8+ T cells. In some embodiments, a mixture of cells is obtained by enriching for CD4+ and CD8+ T cells, yielding an enriched mixture of CD4+ and CD8+ cells. In certain embodiments, the mixture of cells comprises 1-10%, 1-20%, 1-30%, 1-40%, 1-50%, 1-60%, 10-20%, 10-30%, 10-40%, 10-50%, 10-60%, 20-30%, 20-40%, 20-50%, 20-60%, 30-40%, 30-50%, or 30-60% of CD8⁺ T cells (e.g., CD8⁺ cytotoxic T cells) out of all T cells in the population. In certain embodiments, the mixture of cells further comprises 1-10%, 1-20%, 1-30%, 1-40%, 1-50%, 1-60%, 1-70%, 10-20%, 10-30%, 10-40%, 10-50%, 10-60%, 10-70%, 20-30%, 20-40%, 20-50%, 20-60%, 20-70%, 30-40%, 30-50%, 30-60%, or 30-70% of CD4⁺ T cells (e.g., CD4⁺ helper T cells) out of all T cells in the population. In certain embodiments, the mixture of cells comprise CD8⁺ T cells (e.g., CD8⁺ cytotoxic T cells) and CD4⁺ T cells (e.g., CD4⁺ helper T cells) at a ratio of 1:5 to 5:1, 1:4 to 4:1, 1:3 to 3:1, 1:2 to 2:1, 1:5 to 2:1, 1:4 to 2:1, 1:3 to 1:1, or 1:2 to 1:1. In some embodiments, the mixture of cells comprise CD8+ T cells and CD4+ T cells at a ratio of about 1:2. In some embodiments, a host cell or mixture of host cells are expanded before introduction of an engineered nucleic acid or vector or plasmid comprising an engineered nucleic acid. In some embodiments, a host cell or mixture of host cells are allogeneic. In some embodiments, a host cell or mixture of host cells are autologous.

In some embodiments, introducing an engineered nucleic acid (or a vector or plasmid comprising an engineered nucleic acid) to a host cell comprises contacting the host cell with a viral vector (e.g., any viral vector described herein). In some embodiments, a viral vector is selected from the group consisting of: a lentiviral vector, a retroviral vector, an adenoviral vector, transposons, cosmids, and an AAV vector. In some embodiments, a viral vector is a lentiviral vector. In some embodiments, a step of introducing an engineered nucleic acid (or a vector or plasmid comprising an engineered nucleic acid) to a host cell comprises use of viral transduction. Any known method of introducing nucleic acids (including nucleic acid vectors and plasmids) into a host cell may be used in accordance with the present disclosure.

Methods of introducing nucleic acid constructs into a cell (e.g., a eukaryotic cell) are known in the art. Non-limiting examples of methods that can be used to introduce an engineered nucleic acid or nucleic acid construct (e.g., a vector or plasmid comprising an engineered nucleic acid) into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection. As used herein, “transformed” and “transduced” are used interchangeably.

In some embodiments, an engineered nucleic acid is introduced to a cell using a lentiviral vector. In some embodiments, the lentiviral vector is used at a multiplicity of infection (MOI) of about 1, about 2, about 3, about 4, about 5, about 6, about 7, about 8, about 9, about 10, or greater. In some embodiments, the lentiviral vector is used at an MOI of about 1. In some embodiments, the lentiviral vector is used at an MOI of about 2. In some embodiments, the lentiviral vector is used at an MOI of about 3. In some embodiments, the lentiviral vector is used at an MOI of about 4. In some embodiments, the lentiviral vector is used at an MOI of about 5. In some embodiments, the lentiviral vector is used at an MOI of about 6. In some embodiments, the lentiviral vector is used at an MOI of about 7. In some embodiments, the lentiviral vector is used at an MOI of about 8. In some embodiments, the lentiviral vector is used at an MOI of about 9. In some embodiments, the lentiviral vector is used at an MOI of about 10.

In some embodiments, a provided method further includes a step of contacting a host T cell with an effective amount of one or more CD3-stimulation agents in the absence of a CD28 stimulating agent under conditions that allow for the stimulation of the host T cell. In other embodiments, a provided method further includes a step of contacting a host T cell with an effective amount of one or more agents that activate both CD3 and CD28 (e.g., a solid surface, such as a polymeric nanomatrix, coated with an anti-CD3 antibody and an anti-CD28 antibody) under conditions that allow for the stimulation of the host T cell.

In some embodiments, the present disclosure provides a method of making an engineered T cell, the method comprising steps of: (a) obtaining a host T cell from a subject; and (b) introducing an engineered nucleic acid (e.g., any engineered nucleic acid described herein) to the host T cell. In some embodiments, a method of making an engineered T cell further comprises a step of contacting a host T cell with an effective amount of one or more CD3-stimulation agents in the absence of a CD28 stimulating agent under conditions that allow for the stimulation of the host T cell. In other embodiments, a method of making an engineered T cell further comprises a step of contacting a host T cell with an effective amount of one or more agents that activate both CD3 and CD28 (e.g., a magnetic bead coated with an anti-CD3 antibody and an anti-CD28 antibody) under conditions that allow for the stimulation of the host T cell. In certain embodiments, the stimulation step is taken prior to step (b).

In some embodiments, the present disclosure provides a method of making an engineered T cell, the method comprising steps of: (a) obtaining a host T cell from a subject; and (b) introducing an engineered nucleic acid (e.g., any engineered nucleic acid described herein, e.g., a vector comprising an engineered nucleic acid, using any method of introducing provided herein) to the host T cell.

In some embodiments, the present disclosure provides a method of making an engineered T cell, the method comprising steps of: (a) obtaining a host T cell mixture from a subject; and (b) introducing an engineered nucleic acid (e.g., any engineered nucleic acid described herein, e.g., a vector comprising an engineered nucleic acid, using any method of introducing provided herein) to the host T cell mixture. In some embodiments, a method of making an engineered T cell further comprises a step of enriching a host T cell mixture for CD4+ and CD8+ T cells prior to introducing an engineered nucleic acid to the host T cell mixture.

In some embodiments, a method of making an engineered T cell further comprises a step of expanding a host T cell or T cell mixture before introduction of an engineered nucleic acid or vector or plasmid comprising an engineered nucleic acid. In certain embodiments, the T cells are expanded after introduction of the engineered nucleic acid, while the nucleic acid is still in the cell culture medium. In certain embodiments, the T cells are expanded for at least 3, 4, 5, 6, 7, or 8 days in the presence of one or more cytokines, including but not limited to IL-2, IL-7, and/or IL-15. In certain embodiments, the T cells are expanded for at least 3, 4, 5, 6, 7, or 8 days in the presence of IL-7 and IL-15.

Also provided herein are engineered T cells produced using any of the methods described herein. The present disclosure provides for engineered T cells comprising an engineered nucleic acid (e.g., any of the engineered nucleic acid described herein). In many embodiments of the present disclosure an engineered T cell comprises an engineered nucleic acid encoding a CAR (e.g., any CAR described herein). In some embodiments, the present disclosure provides an engineered T cell comprising a nucleic acid sequence encoding an amino acid sequence as set forth in SEQ ID NO: 13. In some embodiments, the present disclosure provides an engineered T cell comprising an engineered nucleic acid sequence as set forth in SEQ ID NO: 14.

The population of engineered T cells produced may include T cells of various phenotypes, such as naive T (T_{N}) cells characterized as CD45RO−, CCR7+, and CD95−, central memory T (T_{CM}) cells characterized as CD45RO+ and CCR7+, effector memory T (T_{EM}) cells characterized as CD45RO+ and CCR7−, stem memory T (T_{SCM}) cells characterized as CD45RO−, CCR7+, and CD95+, and effector memory cells re-expressing CD45RA T (T_{EMRA}) cells characterized as CD45RO− and CCR7−. Alternative characterizations of these T cell subsets include but are not limited to naive T (T_{N}) cells characterized as CD45RA+, CCR7+, and CD95−, central memory T (T_{CM}) cells characterized as CD45RA− and CCR7+, effector memory T (T_{EM}) cells characterized as CD45RA− and CCR7−, stem memory T (T_{SCM}) cells characterized as CD45RA+, CCR7+, and CD95+, and effector memory cells re-expressing CD45RA T (T_{EMRA}) cells characterized as CD45RA+ and CCR7−. In some embodiments, the population comprises at least 25%, 30%, 40%, 50%, 60%, 70%, 75%, or 80% CD4+ T cells, out of all T cells in the population. In some embodiments, the population comprises at least 20%, 25%, 30%, 40%, 50%, 55%, or 60% CD8+ T cells, out of all T cells in the population. In some embodiments, the population comprises at least 0.05%, 0.1%, 0.2%, 0.3%, 0.4%, 0.5%, 1%, 2%, 3%, 4%, 5%, 6%, or 7% CD4+T_{N}, out of all T cells in the population. In some embodiments, the population comprises at least 0.05%, 0.1%, 0.2%, 0.3%, 0.4%, 0.5%, 0.6%, 0.7%, 0.8%, 0.9%, 1%, 5%, 10%, 15%, 20%, 25%, 30%, or 35% CD4+T_{SCM}, out of all T cells in the population. In some embodiments, the population comprises at least 10%, 15%, 20%, 25%, or 30% CD4+T_{CM}, out of all T cells in the population. In some embodiments, the population comprises at least 20%, 25%, 30%, 35%, 40%, 45%, 50%, 55%, 60%, 65%, 70%, 75%, 80%, or 85% CD4+T_{EM}, out of all T cells in the population. In some embodiments, the population comprises at least 5%, 10%, 15%, 20%, 25%, or 30% CD4+T_{EMRA}, out of all T cells in the population. In some embodiments, the population comprises at least 0.05%, 0.1%, 0.2%, 0.3%, 0.4%, 0.5%, 1%, 5%, 10%, 15%, 20%, or 25% CD8+T_{N}, out of all T cells in the population. In some embodiments, the population comprises at least 0.05%, 0.1%, 0.2%, 0.3%, 0.4%, 0.5%, 0.6%, 0.7%, 0.8%, 0.9%, 1%, 5%, 10%, 15%, 20%, 25%, 30%, 35%, 40%, 45%, 50%, 55%, or 60% CD8+T_{SCM}, out of all T cells in the population. In some embodiments, the population comprises at least 0.05%, 0.1%, 0.2%, 0.3%, 0.4%, 0.5%, 0.6%, 0.7%, 0.8%, 0.9%, 1%, 5%, 10%, 15%, 20%, 25%, or 30% CD8+T_{CM}, out of all T cells in the population. In some embodiments, the population comprises at least 5%, 10%, 15%, 20%, 25%, 30%, 35%, 40%, 45%, or 50% CD8+T_{EM}, out of all T cells in the population. In some embodiments, the population comprises at least 5%, 6%, 7%, 8%, 9%, 10%, 15%, 20%, 25%, 30%, 35%, 40%, 45%, 50%, 55%, or 60% CD8+T_{TEMRA}, out of all T cells in the population. It will be understood by a skilled artisan that the percentage of certain T cells present in a population of engineered T cells may vary from patient to patient.

### Compositions

Also provided herein are compositions (e.g., pharmaceutical compositions) comprising any engineered T cell described herein or any engineered nucleic acid described herein. In some embodiments, a provided pharmaceutical compositions can be formulated for intravenous administration. In some embodiments, a pharmaceutical compositions can include a pharmaceutically acceptable carrier (e.g., phosphate buffered saline).

### Kits

Also provided herein are kits that include any of the compositions described herein. For example, a kit can include one or more of any of the nucleic acid constructs described herein. In other examples, a kit can include any of engineered T cell described herein or one or more doses of a composition including any engineered T cell described herein. In some embodiments, a kit can include instructions for performing any of the methods described herein.

### Methods of Treatment

Provided herein are methods and compositions for reducing the number of B cells in a tissue in a subject having an autoimmune disease. In some embodiments, the present disclosure provides a method of reducing the number of B cells in a tissue in a subject having an autoimmune disease, the method comprising administering a therapeutically effective amount of any engineered T cell described herein to the subject. Also provided herein are methods of treating a subject having an autoimmune disease (e.g., a B cell-associated autoimmune disease). In some embodiments, the present disclosure provides for a method of treating a subject having an autoimmune disease, the method comprising administering a therapeutically effective amount of an engineered T cell (e.g., any engineered T cell described herein, e.g., an anti-CD19 CAR T cell) to the subject.

In some embodiments, a B cell-associated autoimmune disease or disorder is systemic lupus erythematosus. In some embodiments, a B cell-associated autoimmune disease or disorder is lupus nephritis. In some embodiments, a B cell-associated autoimmune disease or disorder is Class III or Class IV lupus nephritis. In some embodiments, a B cell-associated autoimmune disease or disorder is Class III lupus nephritis. In some embodiments, a B cell-associated autoimmune disease or disorder is Class IV lupus nephritis. In some embodiments, a B cell-associated autoimmune disease or disorder is Class II lupus nephritis.

In some embodiments, a subject receiving a provided treatment (e.g., any engineered nucleic acid, engineered T cell, or CAR provided herein) has previously been treated with a lymphodepletion agent (e.g., cyclophosphamide and/or fludarabine). In some embodiments, a subject receiving a provided treatment has previously received a standard of care treatment for an autoimmune disease (e.g., a B cell-associated autoimmune disease) that was ineffective and/or caused one or more adverse side effects. For example, in some embodiments, a subject receiving a presently provided treatment has previously been treated with an immunosuppressive drug. In some embodiments, a subject receiving a presently provided treatment has previously been treated with corticosteroids (e.g., glucocorticoids such as betamethasone, dexamethasone, cortisone, hydrocortisone, prednisone, prednisolone, methylprednisolone). In some embodiments, a subject receiving a provided treatment has previously been treated with a B-cell depleting antibody (e.g., an anti-CD20 antibody such as rituximab, an anti-BAFF antibody such as belimumab, etc.). In some embodiments, a subject receiving a provided treatment has previously been treated with a calcineurin inhibitor. In some embodiments, a subject receiving a presently provided treatment has previously been treated with mycophenolate mofetil.

In some embodiments, a subject receiving a presently provided treatment has previously received a standard of care treatment for lupus nephritis (e.g., an immunosuppressive drug), and the standard of care treatment dose is reduced (e.g., by tapering) before T cells are obtained from the subject for making engineered T cells (e.g., via any method described herein). In some embodiments, a standard of care treatment dose is reduced at least 4 weeks, at least 5 weeks, at least 6 weeks, at least 7 weeks, or at least 8 weeks prior to a subject receiving a provided treatment. In some embodiments, a standard of care treatment dose is reduced at least 6 weeks prior to a subject receiving a provided treatment. In some embodiments, a subject continues to receive a standard of care treatment (e.g., at a reduced dose, or keeping only a limited number of individual therapeutic components from the original SOC treatment) while also receiving the presently provided treatment. In some embodiments, a subject does not continue to receive a standard of care treatment while receiving the presently provided treatment.

The present disclosure appreciates that therapeutic regimens for autoimmune diseases or disorders generally require chronic administration or administration of multiple treatment cycles to effectively treat the disease or disorder. The most common treatments include corticosteroids and immunosuppressive drugs, which can be very toxic to a subject. In some cases, these drugs can also suppress a subject's immune system, resulting in serious infections and/or adverse side effects in bone marrow, liver, and/or kidneys. Accordingly, standard of care treatments such as corticosteroid and immunosuppressive drug combinations present challenges to chronic use. In contrast, use of engineered T cells provided herein offers the premise of a single infusion possibly controlling disease for a prolonged period of time. Use of an engineered T cells as provided herein also permits repeated treatments (e.g., chronic administration, multiple treatment cycles, etc.) due to low toxicity profile and subsequent reduced side effects, e.g., as a result of the fully human nature the scFv, among other things. In many embodiments, use of the provided engineered T cells results in lower toxicity with subsequent reduced adverse effects over time and can be repeated in the future as needed.

In some embodiments of any of the methods described herein, a step of administering comprises administering two or more doses of an engineered T cell (e.g., any engineered T cell described herein). In some embodiments, a step of administering comprises administering five or more doses of an engineered T cell. In some embodiments, a step of administering comprises administering ten or more doses of an engineered T cell. In some embodiments, a dose of an engineered T cell is fractionated such that the total dose is administered over the course at least two, three, four, five, six, seven, or more days.

The engineered T cells used in the methods of the present disclosure can be generated by a process disclosed herein. In some embodiments, an engineered T cell is generated by introducing into a T cell an engineered nucleic acid comprising a nucleic acid sequence encoding a CAR (e.g., any CAR described herein, e.g, an anti-CD19 CAR). In some embodiments, an engineered nucleic acid further comprises a promoter operably linked to a nucleic sequence encoding a CAR. In some embodiments, an engineered T cell is generated by further contacting the T cell with an effective amount of one or more agents that activate CD3 and CD28 under conditions that allow for stimulation of the T cell. In some embodiments, a T cell is obtained from a subject (e.g., an autologous or allogeneic subject), prior to a step of generating an engineered T cell and a step of administering the engineered T cell. An engineered T cell can be generated or made using any method of making an engineered T cell described herein, e.g., in the “Methods of Making Engineered T Cells” section above.

In some embodiments, the present disclosure provides for a method of reducing the number of B cells in a tissue in a subject having an autoimmune disease, the method comprising a step of administering an engineered T cell (e.g., any engineered T cell described herein). In some embodiments, the present disclosure provides for a method of reducing the number of B cells in a tissue in a subject having an autoimmune disease, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating lupus nephritis, the method comprising a step of administering an engineered T cell (e.g., any engineered T cell described herein). In some embodiments, the present disclosure provides for a method of treating lupus nephritis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class III lupus nephritis, the method comprising a step of administering an engineered T cell (e.g., any engineered T cell described herein). In some embodiments, the present disclosure provides for a method of treating Class III lupus nephritis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class IV lupus nephritis, the method comprising a step of administering an engineered T cell (e.g., any engineered T cell described herein). In some embodiments, the present disclosure provides for a method of treating Class IV lupus nephritis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, the present disclosure provides for a method of treating Class II lupus nephritis, the method comprising a step of administering an engineered T cell (e.g., any engineered T cell described herein). In some embodiments, the present disclosure provides for a method of treating Class II lupus nephritis, the method comprising a step of administering an engineered T cell, wherein the engineered T cell is an anti-CD19 CAR T cell. In some embodiments, an anti-CD19 CAR T cell comprises a nucleic acid encoding an amino acid sequence as set forth in SEQ ID NO: 13. In some embodiments, an anti-CD19 CAR T cell comprises a nucleic acid as set forth in SEQ ID NO: 14.

In some embodiments, provided engineered T cells are administered using parenteral administration (e.g., intravenous administration). Methods and compositions of the present disclosure may be administered using any suitable method.

In some embodiments, administering of methods and compositions provided herein to a subject with an autoimmune disease or disorder results in amelioration of one or more symptoms of the autoimmune disease or disorder in the subject. In some embodiments, administering of methods and compositions provided herein to a subject with an autoimmune disease or disorder results in a reduction in the number, severity, or frequency of one or more symptoms of the autoimmune disease or disorder in the subject (e.g., as compared to the number, severity, or frequency of the one or more symptoms of the autoimmune disease or disorder in the subject prior to receiving treatment with provided methods or compositions). In some embodiments, a subject having an autoimmune disease having been administered an engineered T cell as described here can experience a reduction in inflammation and/or auto-antibody production.

A pharmaceutical composition containing an engineered T cell and a pharmaceutically acceptable carrier or buffer can be administered to a subject having an autoimmune disease. In some embodiments, a provided pharmaceutical composition to be administered to a subject having an autoimmune disease can be formulated in an injectable form (e.g., as solution and/or suspension). In some embodiments, a pharmaceutical composition comprising an engineered T cell as provided herein can further include phosphate buffered saline. Pharmaceutically acceptable carriers, fillers, and vehicles that can be used in a pharmaceutical composition described herein can include, without limitation, ion exchangers, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, and sodium chloride.

An effective dosage (e.g., for a provided T cell composition) to administer to a patient intravenously can vary depending on the severity of an autoimmune disease, the age and general health condition of a subject, excipient usage, the possibility of co-usage with other therapeutic treatments, and the judgment of the treating physician. An effective amount of an engineered T cell can be any amount that reduces inflammation and auto-antibody production within a subject having an autoimmune disease (e.g., via deletion or reduction of autoreactive B cells) without producing significant toxicity to the subject. In many embodiments, an effective dosage may also be dependent on the level of CAR expression in the provided engineered T cells and/or the percentage of engineered T cells within a provided composition. In some cases, engineered T cells can be a purified population of engineered T cells generated as described herein. In some cases, the purity of a population of engineered T cells can be assessed using any appropriate method, including, without limitation, flow cytometry. In some embodiments, purity of a population of engineered T cells can be assessed by quantifying the amount of T cells expressing the CAR relative to all the T cells in the population. In some cases, a population of engineered T cells to be administered to a subject can include a range of purities from about 5% to about 80%, about 10% to about 80%, about 15% to about 80%, about 20% to about 80%, about 30% to about 80%, about 40% to about 80%, about 50% to about 80%, about 60% to about 80%, about 5% to about 70%, about 5% to about 60%, about 5% to about 50%, about 5% to about 40%, about 5% to about 30%, about 5% to about 20%, about 5% to about 10%, about 70% to about 100%, from about 70% to about 90%, from about 70% to about 80%, from about 80% to about 90%, from about 90% to about 100%, from about 80% to about 100%, from about 80% to about 90%, or from about 90% to 100%. In some cases, a dosage of a provided therapy (e.g., number of engineered T cells to be administered) can adjusted based on the level of purity of the therapy.

In some embodiments, provided compositions (e.g., pharmaceutical compositions) of engineered T cells are administered at a dose of about 0.1×10⁸, about 0.2×10⁸, about 0.3×10⁸, about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, or about 1.5×10⁸ engineered cells. In some embodiments, provided compositions (e.g., pharmaceutical compositions) of engineered T cells are administered at a dose of about 0.5×10⁸ engineered cells. In some embodiments, provided compositions (e.g., pharmaceutical compositions) of engineered T cells are administered at a dose of about 1.0×10⁸ engineered cells.

The frequency of administration of an engineered T cell can be any frequency that reduces inflammation or auto-antibody production within a subject having an autoimmune disease (e.g., via deletion or reduction of autoreactive B cells) without producing toxicity to the subject. In some embodiments, the actual frequency of administration can vary depending on various factors including, without limitation, the effective amount, duration of treatment, use of multiple treatment agents, and severity of the condition may require an increase or decrease in frequency of administration.

An effective duration for administering a composition containing an anti-CD19 CAR T cell or a nucleic acid encoding the same can be any duration that reduces inflammation or auto-antibody production within the subject having an autoimmune disease (e.g., via deletion or reduction of autoreactive B cells) without producing toxicity to the subject. In some embodiments, the effective duration can vary from several days to several months. In some embodiments, the effective treatment duration for administering a composition containing an engineered T cell to treat an autoimmune disease can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some embodiments, the effective treatment duration is at least one year, two years, three years, or more. In some embodiments, a subject receives an infusion of a provided treatment and is cured (e.g., via initiation of an immune reset).

In some embodiments, a course of treatment and/or the severity of one or more symptoms related to autoimmune disease can be monitored. Any appropriate method can be used to determine whether the autoimmune disease is being treated. For example, immunological techniques (e.g., ELISA) can be performed to determine if the level of auto-antibodies present within the subject being treated as described herein is reduced following the administration of an engineered T cell. Remission and relapse of the disease can be monitored by testing for one or more markers of the autoimmune disease.

Any appropriate autoimmune disease or disorder can be treated with an engineered T cell as described herein. In some cases, an autoimmune disease or disorder is caused by the accumulation of auto-antibodies and can be treated with an engineered T cell as described herein.

For continued efficacy, a subject can receive 1, 2, 3, 4, 5, 6, 7, 8, 9, 10, 11, 12, 13, 14, 15, 16, 17, 18, 19, 20, 30, 40, 50, or more doses of any engineered T cell described herein. In some embodiments, a subject receives at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 doses of an engineered T cell (e.g., a T cell expressing a CAR, e.g., a CAR comprising an amino acid sequence as set forth in SEQ ID NO: 13).

In some embodiments, administration of a provided method and composition results in a reduction (e.g., at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction, about a 1% reduction to about a 90% reduction, about a 1% reduction to about a 80% reduction, about a 1% reduction to about a 70% reduction, about a 1% reduction to about a 60% reduction, about a 1% reduction to about a 50% reduction, about a 1% reduction to about a 45% reduction, about a 1% reduction to about a 40% reduction, about a 1% reduction to about a 35% reduction, about a 1% reduction to about a 30% reduction, about a 1% reduction to about a 25% reduction, about a 1% reduction to about a 20% reduction, about a 1% reduction to about a 15% reduction, about a 1% reduction to about a 10% reduction, about a 1% reduction to about a 5% reduction, about a 5% reduction to about a 99% reduction, about a 5% reduction to about a 90% reduction, about a 5% reduction to about a 80% reduction, about a 5% reduction to about a 70% reduction, about a 5% reduction to about a 60% reduction, about a 5% reduction to about a 50% reduction, about a 5% reduction to about a 45% reduction, about a 5% reduction to about a 40% reduction, about a 5% reduction to about a 35% reduction, about a 5% reduction to about a 30% reduction, about a 5% reduction to about a 25% reduction, about a 5% reduction to about a 20% reduction, about a 5% reduction to about a 15% reduction, about a 5% reduction to about a 10% reduction, about a 10% reduction to about a 99% reduction, about a 10% reduction to about a 90% reduction, about a 10% reduction to about a 80% reduction, about a 10% reduction to about a 70% reduction, about a 10% reduction to about a 60% reduction, about a 10% reduction to about a 50% reduction, about a 10% reduction to about a 45% reduction, about a 10% reduction to about a 40% reduction, about a 10% reduction to about a 35% reduction, about a 10% reduction to about a 30% reduction, about a 10% reduction to about a 25% reduction, about a 10% reduction to about a 20% reduction, about a 10% reduction to about a 15% reduction, about a 15% reduction to about a 99% reduction, about a 15% reduction to about a 90% reduction, about a 15% reduction to about a 80% reduction, about a 15% reduction to about a 70% reduction, about a 15% reduction to about a 60% reduction, about a 15% reduction to about a 50% reduction, about a 15% reduction to about a 45% reduction, about a 15% reduction to about a 40% reduction, about a 15% reduction to about a 35% reduction, about a 15% reduction to about a 30% reduction, about a 15% reduction to about a 25% reduction, about a 15% reduction to about a 20% reduction, about a 20% reduction to about a 99% reduction, about a 20% reduction to about a 90% reduction, about a 20% reduction to about a 80% reduction, about a 20% reduction to about a 70% reduction, about a 20% reduction to about a 60% reduction, about a 20% reduction to about a 50% reduction, about a 20% reduction to about a 45% reduction, about a 20% reduction to about a 40% reduction, about a 20% reduction to about a 35% reduction, about a 20% reduction to about a 30% reduction, about a 20% reduction to about a 25% reduction, about a 25% reduction to about a 99% reduction, about a 25% reduction to about a 90% reduction, about a 25% reduction to about a 80% reduction, about a 25% reduction to about a 70% reduction, about a 25% reduction to about a 60% reduction, about a 25% reduction to about a 50% reduction, about a 25% reduction to about a 45% reduction, about a 25% reduction to about a 40% reduction, about a 25% reduction to about a 35% reduction, about a 25% reduction to about a 30% reduction, about a 30% reduction to about a 99% reduction, about a 30% reduction to about a 90% reduction, about a 30% reduction to about a 80% reduction, about a 30% reduction to about a 70% reduction, about a 30% reduction to about a 60% reduction, about a 30% reduction to about a 50% reduction, about a 30% reduction to about a 45% reduction, about a 30% reduction to about a 40% reduction, about a 30% reduction to about a 35% reduction, about a 35% reduction to about a 99% reduction, about a 35% reduction to about a 90% reduction, about a 35% reduction to about a 80% reduction, about a 35% reduction to about a 70% reduction, about a 35% reduction to about a 60% reduction, about a 35% reduction to about a 50% reduction, about a 35% reduction to about a 45% reduction, about a 35% reduction to about a 40% reduction, about a 40% reduction to about a 99% reduction, about a 40% reduction to about a 90% reduction, about a 40% reduction to about a 80% reduction, about a 40% reduction to about a 70% reduction, about a 40% reduction to about a 60% reduction, about a 40% reduction to about a 50% reduction, about a 40% reduction to about a 45% reduction, about a 45% reduction to about a 99% reduction, about a 45% reduction to about a 90% reduction, about a 45% reduction to about a 80% reduction, about a 45% reduction to about a 70% reduction, about a 45% reduction to about a 60% reduction, about a 45% reduction to about a 50% reduction, about a 50% reduction to about a 99% reduction, about a 50% reduction to about a 90% reduction, about a 50% reduction to about a 80% reduction, about a 50% reduction to about a 70% reduction, about a 50% reduction to about a 60% reduction, about a 60% reduction to about a 99% reduction, about a 60% reduction to about a 90% reduction, about a 60% reduction to about a 80% reduction, about a 60% reduction to about a 70% reduction, about a 70% reduction to about a 99% reduction, about a 70% reduction to about a 90% reduction, about a 70% reduction to about a 80% reduction, about a 80% reduction to about a 99% reduction, about a 80% reduction to about a 90% reduction, or about a 90% reduction to about a 99% reduction) in the number of B cells in a tissue of the subject (e.g., in peripheral blood) having an autoimmune disease, e.g., as compared to the levels in the subject prior to treatment or the levels in a similar subject not treated or receiving a different treatment. In some embodiments, after deep measured depletion of B cells in a tissue of a subject (e.g., in peripheral blood), B cell counts may increase and substantially recover to normal levels, e.g., as compared to levels in the subject prior to treatment or the levels in a similar subject not treated or receiving a different treatment or any other suitable control. In some embodiments, B cell counts will substantially recover to normal levels after about 3, 4, 5, 6, 7, 8, 9, 10, 11, or 12 months. In some embodiment, recovered B cells will show a sustained naive phenotype profile, indicating the potential for an immune reset and a long-term functional cure of the subject. Without wishing to be bound by theory, it is contemplated that the B cell depletion caused by the CAR T cells may lead to an immune reset, e.g., as evidenced by the durable absence of symptoms of the disease even in the presence of reconstituted B cell numbers.

In some embodiments, administration of methods and compositions described herein result in a reduction (e.g., at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction (or any of the subranges of this range described herein) in the level of auto-antibodies in the subject having the autoimmune disease, e.g., as compared to the levels in the subject prior to treatment or the levels in a similar subject not treated or receiving a different treatment.

Technologies of the present disclosure will be further described in the following examples, which do not limit the scope of the disclosure described in the claims.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their invention.

### Example 1. Preparation of Engineered T Cells Comprising a Lentivirus Vector Encoding Hu19-CD828Z

A Hu19-CD828Z may be prepared as described in U.S. Pat. No. 10,287,350. Briefly, fully human anti-CD19 CARs were generated by utilizing sequences of the fully human 47G4 monoclonal antibody (described in U.S. Patent Application Publication No. 2010/0104509). The 47G4 antibody was generated by vaccinating mice of the KM strain, which carry a human kappa light chain transgene and a human heavy chain transchromosome. The sequences of the 47G4 antibody light chain and heavy chain variable regions were obtained from U.S. Patent Application Publication No. 2010/0104509. A 47G4 scFv was designed comprising the following elements from 5′ to 3′: a CD8 signal sequence, the 47G4 antibody light chain variable region, a linker sequence (encoding a peptide comprising the amino acid sequence GSTSGSGKPGSGEGSTKG (SEQ ID NO: 9)) (see Cooper et al., Blood, 101(4): 1637-1644 (2003)), and the 47G4 antibody heavy chain variable region. A DNA sequence encoding a CAR was then designed comprising the following components from 5′ to 3′: the 47G4 scFv described above, part of the extracellular region and all of the transmembrane region of the human CD8 molecule, and the cytoplasmic (or intracellular) portions of the human CD28 molecule and the human CD3 zeta. molecule. This CAR was designated 47G4-CD828Z (SEQ ID NO: 13), and the sequence was synthesized by Invitrogen (Carlsbad, Calif.).

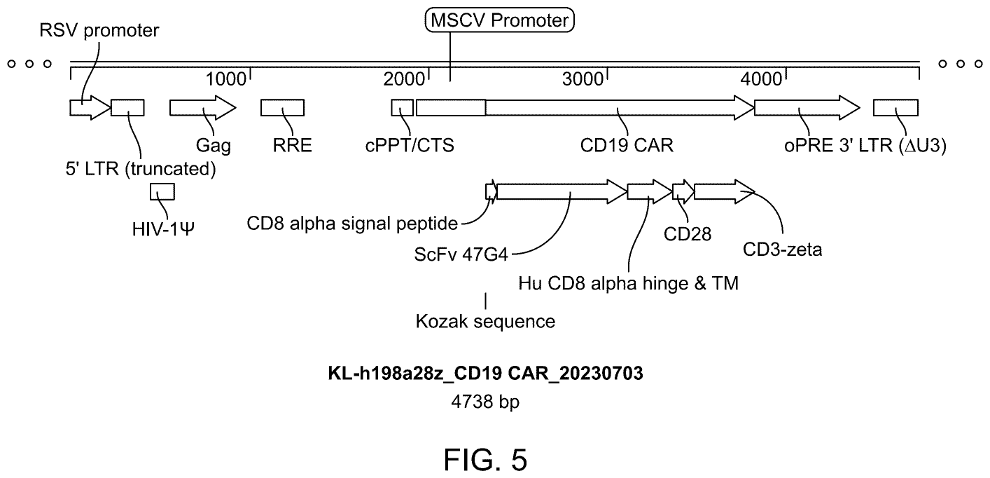

Expression of a Hu19-CD828Z polypeptide in a cell can be performed using any suitable method. In these experiments, T cells (CD4+ T cells, CD8+ T cells, or an cell mixture enriched for CD4+ and CD8+ T cells) are transduced with a lentivirus where the lentiviral vector includes a nucleic acid sequence encoding Hu19-CD828Z polypeptide. The lentiviral vector includes an MSCV promoter among other regulatory factors (see, e.g., FIG. 5). Lentivirus is produced in HEK293 cells according to standard protocols. A KL-h198a28z lentiviral vector system may be used to transduce T cells. KL-h198a28z is a self-inactivating (SIN) vesicular stomatitis virus (VSV)-G pseudotyped 3rd generation lentiviral vector expressing human anti-CD19 chimeric antigen receptor (CAR). The lentiviral vector KL-hl98a28z is manufactured using a HEK 293T cell line transiently transfected with a state-of-the-art four-plasmid system. The envelope protein encoding plasmid (pLTG1292) expresses a heterologous spike protein, the VSV-G protein, under control of the cytomegalovirus (CMV) promoter. The VSV-G envelope protein provides broad cell tropism for transduction of a wide variety of mammalian cell types. KL-h198a28z encodes the CAR construct Hu19-CD828Z, and can be used to manufacture CAR T cells for treating patients with B cell-associated diseases.

Engineered T cells can be produced from blood cells using various known methods, e.g., as described in Ghassemi et al., (2018) Cancer Immunol. Res. 6(9) and Mackensen et al., (2022) Nat. Med. 28:2124-32. In one example, white blood cells are collected from a patient by apheresis. The cells are enriched for CD4+ and CD8+ T cells and are then activated with CD3 and CD28 agonistic agents, e.g., TransAct™, a polymeric nanomatrix coated. Following activation, the cells are transduced with lentiviral vector KL-hl98a28z vector encoding Hu19-CD828Z and expanded in culture. The cells are then harvested and assessed for viability, Hu19-CD828Z expression, T cell phenotype, and potency (e.g., cytotoxicity and cytokine release).

### Example 2. In Vitro Assessment of Systemic Lupus Erythematosus (SLE) Patient-Derived Hu19-CD828Z Transduced Cells

Cytolytic activity and activation of Hu19-CD828Z generated from SLE patient PBMCs against human CD19⁺ malignant B cell cancer cells and autologous SLE patient primary B cells were assessed.

Briefly, three lots of Hu19-CD828Z cells were generated from SLE patient peripheral blood mononuclear cells (PBMCs), two lots of Hu19-CD828Z cells were generated from healthy donor (HD) PBMCs, or untransduced T cells from the same donors were co-cultured overnight with target cells. Target cells were either the human ALL cell line NALM6 (known to express high levels of CD19), autologous (i.e., donor matched) primary B cells that express CD19, and/or the human chronic myeloid leukemia (CML) cell line K562 that does not express CD1, at Effector:Target (E:T) ratios ranging from 0:1 to 3:1 for NALM6 and K562 and 0:5 to 10:1 for autologous primary B cell co-cultures.

Effector and target cells alone were included as negative controls. After co-incubation, the amount of cytokines in the supernatant and the number of live target cells were quantitated.

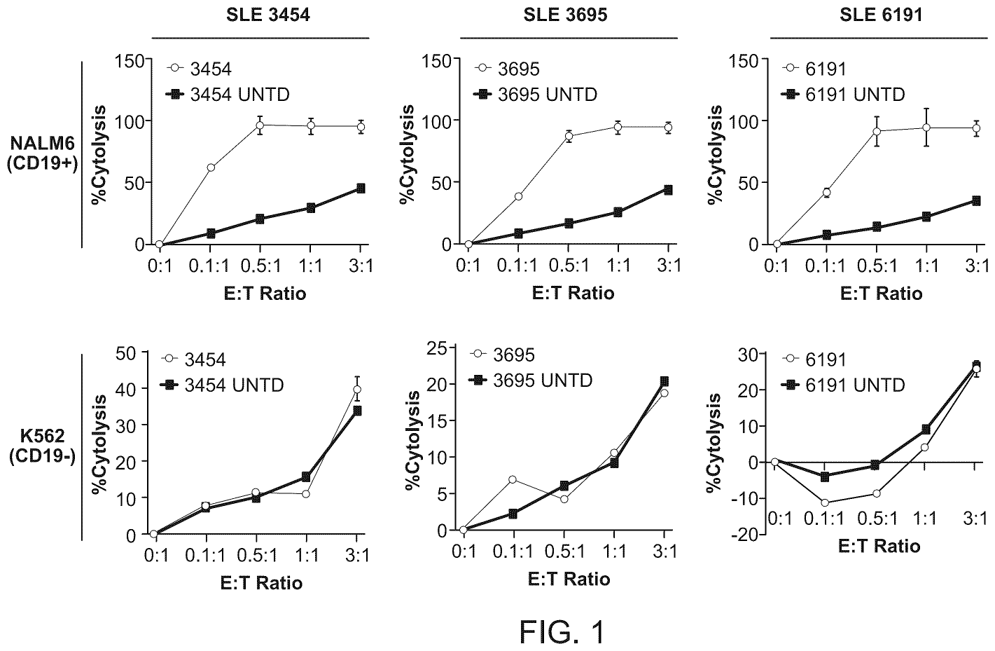

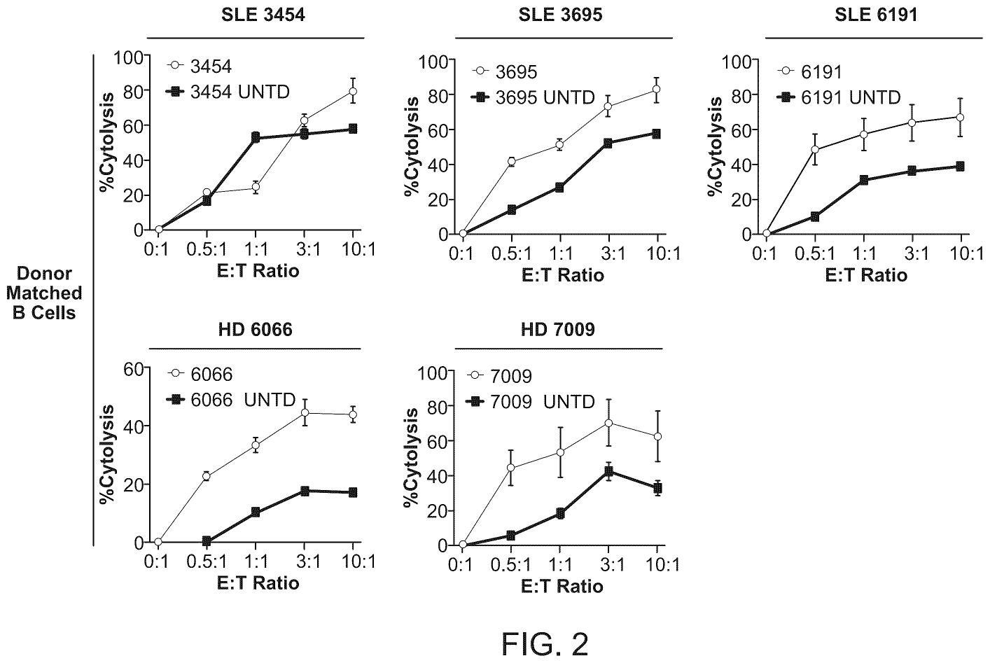

SLE-derived Hu19-CD828Z cells showed CAR-mediated cytotoxicity, cytokine release, and proliferation in a CD19-dependent manner. Cytotoxicity was demonstrated by robust and dose-dependent cytotoxicity against both a CD19⁺ human B cell cancer cell line (NALM6) and minimal cytotoxicity against the K562 CD19⁻ cell line (FIG. 1). SLE-derived Hu19-CD828Z cells from two donors (3695 and 6191) induced strong, dose-dependent cytotoxicity of autologous B cells with the level of cytotoxicity being significantly greater than observed for untransduced T cells, thereby demonstrating that the difference in cytotoxicity is driven by the CAR (FIG. 2).

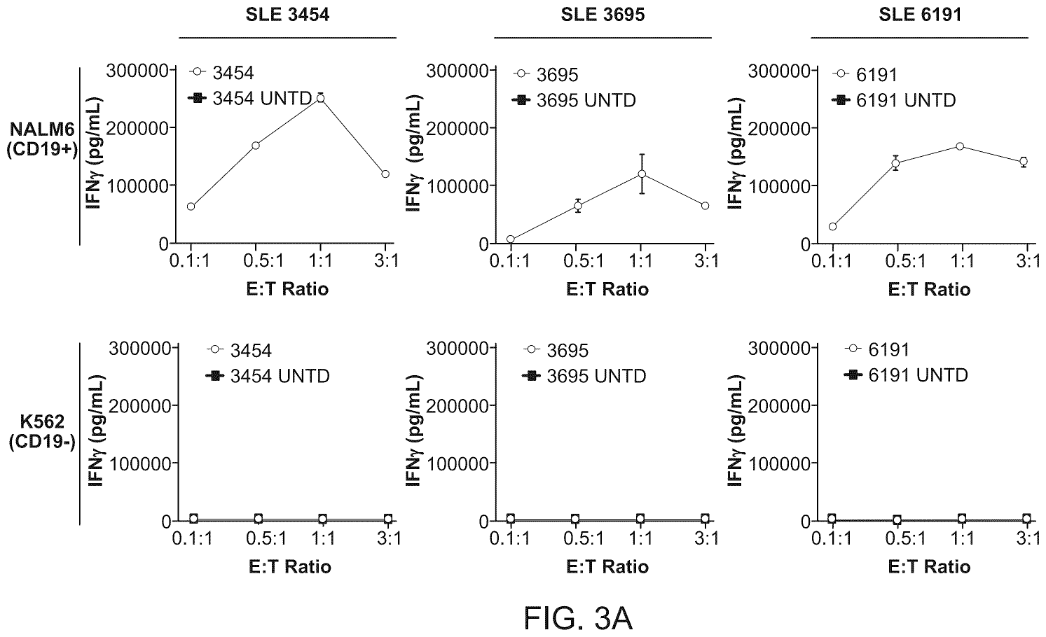

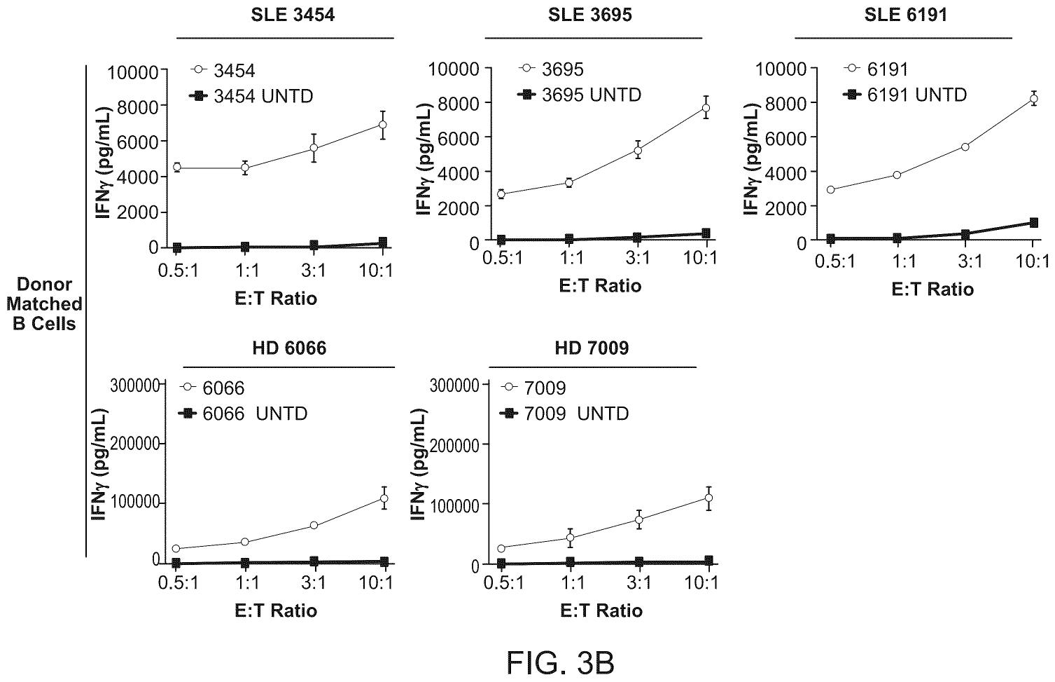

All three lots of SLE-derived Hu19-CD828Z cells also showed CD19-mediated and dose-dependent production of Granzyme B and the cytokines IL-10, IL-13, IL-2, IL-4, IL-8, and TNFα, which are directly related to T cell activation and/or T cell-mediated cytotoxicity, upon Hu19-CCD828Z co-culture with CD19⁺ NALM6 cells and autologous B cells, but not with the CD19− K562 cells (data not shown). Specifically, interferon gamma (IFNγ) was the predominant cytokine detected as shown in FIGS. 3A-3B.

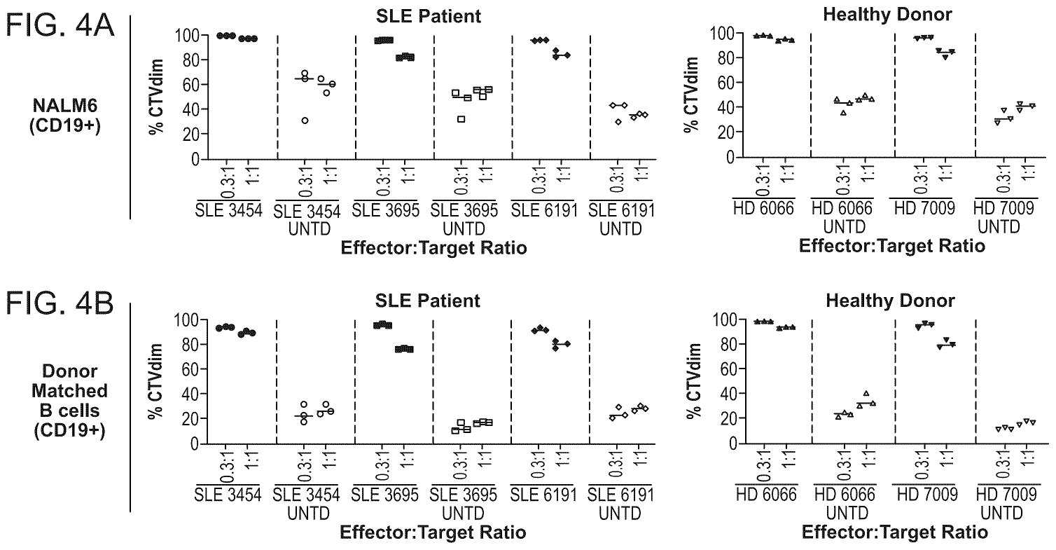

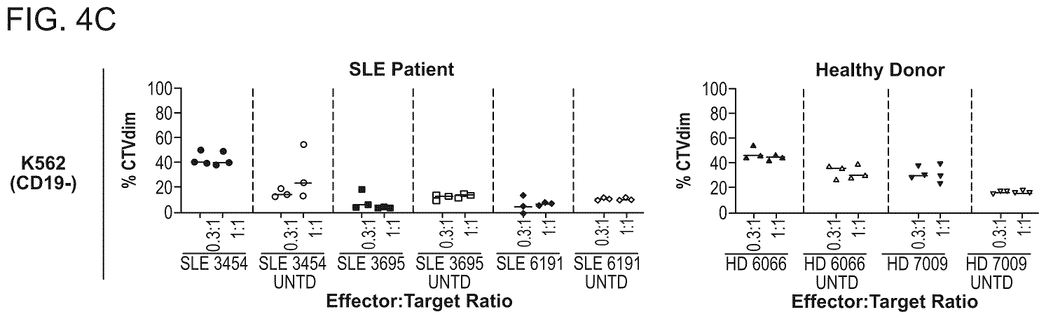

For the target-dependent, CAR-mediated proliferation studies, the same set of SLE- and HD-derived Hu19-CD828Z and untransduced T cells were co-cultured for 96 hours with the same set of CD19⁺ and CD19⁻ target cells (as described herein). Effector and target cells alone were included as negative controls. After co-incubation, the number of Cell Trace Violet (CTV) dim proliferating effector cells was quantified. CD19 was confirmed to be expressed on NALM6 and autologous primary B cells but not expressed on K562 cells (data not shown). SLE-derived Hu19-CD828Z cells also showed CAR-mediated and CD19− mediated proliferation comparable to that from HD-derived Hu19-CD828Z cells (FIGS. 4A-4C).

The data demonstrate that SLE patient-derived Hu19-CD828Z transduced PBMCs exhibit CAR-mediated and CD19-dependent cytotoxicity, cytokine release, and cellular proliferation as demonstrated by activity during co-culture with CD19⁺ cancer cells (NALM6 cells) and autologous primary B cells, but not control CD19⁻ cells (K562). The results from the cytokine release (FIGS. 3A-3B) and proliferation assay (FIGS. 4A-4C) showed clear target-mediated response demonstrating that the activity of the Hu19-CD828Z CAR construct in SLE and Healthy Donor-derived samples is comparable.

### Example 3. Use of Hu19-CD828Z to Treat Lupus Nephritis

This example describes a phase 1 clinical study to assess the safety, tolerability, and clinical activity of KYV-101 (an autologous fully human anti-CD19 CAR T-cell therapy) in adult subjects with refractory lupus nephritis (LN).

Systemic lupus erythematosus (SLE) is a chronic autoimmune disease characterized by a wide spectrum of organ involvement and disease severity. Renal involvement (LN) may occur in approximately 50% of SLE patients. LN is a major risk factor for morbidity and mortality in patients with SLE; 10% of patients with LN will eventually develop end-stage renal disease. Histopathologic classification based on renal biopsy findings can determine the prognosis and treatment approach, with focal and diffuse proliferative LN (Classes III and IV, respectively) requiring intervention with potent immunosuppressive therapies. Despite recent approvals for LN, an unmet need for more efficacious therapies remains with complete renal response (CRR) achieved by only approximately 30-40% of patients over a one-year period with currently available therapies.

B cells play an important role in the pathogenesis of SLE and LN. Autoantibodies to nuclear components (e.g., double-stranded DNA (dsDNA) and Smith (Sm) proteins) produced by B cells are hallmarks of the disease and can form immune complexes that cause tissue damage in target organs, such as kidneys. Diagnostic methods have been developed to detect increased anti-nuclear antibody (ANA), anti-dsDNA antibody, and anti-Sm antibody levels. For example, serologic assays may be performed using enzyme-linked immunosorbent assay to measure the levels of anti-dsDNA and anti-Sm. Further, ANA titer may be determined by indirect immunofluorescence assay on human epithelial type 2 (HEP-2) cells. Efficacy data to date from B cell-targeted therapies strongly support the disease-driving role of B cells in LN, and suggest that much deeper, more sustained depletion of B cells may lead to significantly greater efficacy.

KYV-101 includes autologous CD4+ and CD8+ enriched and expanded T cells genetically engineered to express a CAR that targets CD19, an antigen expressed on the surface of both normal and autoreactive B cells in patients with autoimmune diseases. CD19− targeted CAR T cells harness the ability of cytotoxic T cells to directly and specifically lyse target cells to effectively deplete B cells in the circulation and in lymphoid and potentially non-lymphoid tissues. The CAR used in KYV-101 is Hu19-CD828Z, see, e.g., Example 1.

### Patient Selection

Screening for eligible subjects is performed within 28 days prior to enrollment, based on at least the following inclusion criteria.

Inclusion criteria:
1. Subject must be ≥18 years of age.
2. Clinical diagnosis of SLE according to the 2019 European League Against Rheumatism (EULAR)/American College of Rheumatology (ACR) classification criteria.
3. Active, biopsy-proven proliferative LN Class III or IV according to the 2018 ISN/RPS criteria.
4. Inadequate response to conventional therapies (e.g., at least two conventional therapies).

### Dosage and Administration

KYV-101, is an autologous anti-CD19 CAR T-cell immunotherapy. The KYV-101 coding sequence is composed of a human single-chain variable fragment (scFV) CD19− targeting domain, a CD8 alpha hinge and transmembrane domains (CD8α hinge+TM), a CD28 cytoplasmic costimulatory domain, and a CD3-zeta cytoplasmic domain. The constitutive expression of the CAR is controlled by a murine stem cell virus (MSCV) promoter. KYV-101 is a cell suspension formulated in a chemically defined freezing medium. The finished product is filled in a freezing bag and stored at ≤−150° C. in the vapor phase of liquid nitrogen. In this study, the KYV-101 anti-CD19 CAR T cell immunotherapy is administered at doses of about 1×10⁸ CAR+ T cells and 0.5×10⁸ CAR+ T cells, after a standard lymphodepleting chemotherapy regimen consisting of 300 mg/m²/day of cyclophosphamide and 30 mg/m²/day of fludarabine for 3 days starting on Day −7 to −5. The CAR T cell therapy is administered intravenously as a single infusion on Day 0.

In this study, nine to twelve subjects are enrolled in a standard 3+3 dose-escalation study to evaluate the safety and tolerability of KYV-101. Subjects are enrolled sequentially to the dose levels shown in Table 1, according to dose-limiting toxicity (DLT) observed with existing patients.

TABLE 1
Dosing Cohorts

| Dose Level | Total KYV-101 CAR+ T cells |
|---|---|
| 1 | 0.5 × 10⁸ |
| 2 | 1 × 10⁸ |

Preliminary Results

The first patient enrolled in the study, an 18-year-old female patient diagnosed with SLE at age 9, was treated with KYV-101 at Dose Level 1 (0.5×10⁸ CAR T cells), with 56 days of follow-up reported herein. The patient had Class IV LN with persistent proteinuria despite treatment with mycophenolate mofetil, cyclophosphamide, calcineurin inhibitors, rituximab, belimumab, and glucocorticoids. CAR T-cell manufacturing was successful with 61% CAR expression, 99% purity, and 96% viability. After infusion, CAR T cells rapidly expanded with a peak of 8.6 cells/ml on day 15.

The patient experienced grade 1 cytokine release syndrome consisting of fever on days 5 and 6, which responded to acetaminophen. No immune effector cell-associated neurotoxicity syndrome, dose-limiting toxicities (DLTs), or serious adverse events (AEs) occurred. Depletion of B cells was achieved after CAR T-cell infusion and evidence of clinical improvement was observed (Table 2). Absolute neutrophil counts, hemoglobin, and platelets recovered to normal levels by day 14-56. C-reactive protein values normalized by day 14. Anti-dsDNA decreased slightly though remained strongly positive, while complement levels (C3, C4) increased from low to normal levels. Proteinuria (UPCR) improved from 1.5 to 0.5 from day 0 to day 56, and SLEDAI-2K score improved from 22 to 8. Ig concentrations decreased with greater proportional reductions in IgA and IgM than IgG. After CAR T-cell therapy, the patient discontinued immunosuppressive therapy except 10 mg prednisone, which was discontinued on day 31.

TABLE 2
Clinical Outcomes for Patient 1 Through Day 56 After CAR T-Cell Infusion

| Parameter, units [reference range] | Pre-LD | Day 0 | Day 14 | Day 28 | Day 56 |
|---|---|---|---|---|---|
| CD19+ B cells, cells/μL [107-698] | 22 | 0 | 0 | 1 | 7 |
| ANC, ×10⁹/L [1.80-7.00] | 4.5 | 0.5 | 3.7 | 3.6 | 3.8 |
| Hemoglobin, g/dL [11.5-15.5] | 12 | 9.1 | 9.5 | 11.7 | 12.6 |
| Platelets, ×10⁹/L [150-400] | 93 | 54 | 78 | 103 | 175 |
| CRP, mg/L [0-10] | 83.3 | 67.5 | 4.8 | 2.8 | 11.8 |
| Anti-dsDNA, IU [<25 = Negative] | 297 | 294 | 356 | 304 | 216 |
| C3, mg/dL [83-193] | 54 | 59 | 65 | 94 | 135 |
| C4, mg/dL [15.0-57.0] | 9.2 | 13 | 18.7 | 28.8 | 44.2 |
| Proteinuria, [UPCR] | 1.4 | 1.5 | 0.6 | 0.9 | 0.5 |
| SLEDAI-2K | 19 | 22 | 14 | 13 | 8 |
| IgG, mg/dL [757-1941] | 620 | 546 | 452 | 429 | 291 |
| IgM, mg/dL [33-393] | 87 | 73 | 23 | 12 | 5 |
| IgA, mg/dL [87-534] | 118 | 113 | 62 | 46 | 37 |

ANC, absolute neutrophil count; C3, complement component 3; C4, complement component 4; CRP, C-reactive protein; dsDNA, double-stranded DNA; Ig, immunoglobulin; LD, lymphodepletion; SLEDAI-2K, Systemic Lupus Erythematosus Disease Activity Index 2000; UPCR, Urine Protein Creatinine Ratio.

These data demonstrate that KYV-101 was well tolerated with evidence of clinical improvement and further confirm the potential of using anti-CD19 CAR T-cell therapy in treating patients with LN.

### Example 4. Use of Hu19-CD828Z to Treat Lupus Nephritis

This example describes a phase 1/2 clinical study to assess the safety, tolerability, and clinical activity of KYV-101 in adult subjects with refractory lupus nephritis (LN). The KYV-101 therapy used in this clinical study is the same as the one described in Example 3.

### Patient Selection

Screening for eligible subjects is performed within 28 days prior to enrollment, based on at least the following inclusion criteria.

Inclusion criteria:
1. Subject must be ≥18 years of age.
2. Clinical diagnosis of SLE according to the 2019 European League Against Rheumatism (EULAR)/American College of Rheumatology (ACR) classification criteria.
3. Active, biopsy-proven, proliferative LN Class III or IV according to the 2018 ISN/RPS criteria.
4. Inadequate response to conventional therapies.

### Dosage and Administration

For the Phase 1 study, 6-12 subjects are enrolled in a single dose study to evaluate the safety and tolerability of KYV-101. The first three subjects are enrolled sequentially at a dose of 1×10⁸ CAR+ T cells. The safety and tolerability of KYV-101 are assessed.

The Phase 2 study is initiated after the recommended Phase 2 dose (RP2D) has been identified in Phase 1. The RP2D will demonstrate acceptable safety with evidence of sufficient T-cell expansion, B-cell depletion, and other PD activity from at least 28 days for all dosed subjects. The Phase 2 dose expansion portion is designed to evaluate the safety, tolerability, and clinical efficacy of KYV-101 in subjects with refractory LN.

### OTHER EMBODIMENTS

It is to be understood that while the provided technologies have been described in conjunction with the detailed description thereof, the foregoing description is intended to illustrate and not limit the scope of the disclosure, which is defined by the scope of the appended claims. Other aspects, advantages, and modifications are within the scope of the following claims.

SEQUENCE TABLE

| | | SEQ |
|---|---|---|
| Construct | Sequence | ID NO |
| Hu19-CD828Z Heavy | SYAIS | 1 |
| Chain CDR1 (Kabat) | | |
| Hu19-CD828Z Heavy | GIIPIFGTTNYAQQFQG | 2 |
| Chain CDR2 (Kabat) | | |
| Hu19-CD828Z Heavy | EAVAADWLDP | 3 |
| Chain CDR3 (Kabat, | | |
| Chothia) | | |
| Hu19-CD828Z Light | RASQSVSSSYLA | 4 |
| Chain CDR1 (Kabat, | | |
| Chothia) | | |
| Hu19-CD828Z Light | GASSRAT | 5 |
| Chain CDR2 (Kabat, | | |
| Chothia) | | |
| Hu19-CD828Z Light | QQYGSSRFT | 6 |
| Chain CDR3 (Kabat, | | |
| Chothia) | | |
| Hu19-CD828Z Heavy | QVQLVQSGAEVKKPGSSVKVSCKDSGGTFSSYAIS | 7 |
| Chain Variable Region | WVRQAPGQGLEWMGGIIPIFGTTNYAQQFQGRVTI | |
| | TADESTSTAYMELSSLRSEDTAVYYCAREAVAAD | |
| | WLDPWGQGTLVTVSS | |
| Hu19-CD828Z Light | EIVLTQSPGTLSLSPGERATLSCRASQSVSSSYLAW | 8 |
| Chain Variable Region | YQQKPGQAPRLLIYGASSRATGIPDRFSGSGSGTDF | |
| | TLTISRLEPEDFAVYYCQQYGSSRFTFGPGTKVDIK | |
| Hu19-CD828Z Peptide | GSTSGSGKPGSGEGSTKG | 9 |
| Linker | | |
| Full-length Hu19- | EIVLTQSPGTLSLSPGERATLSCRASQSVSSSYLAW | 10 |
| CD828Z CAR without | YQQKPGQAPRLLIYGASSRATGIPDRESGSGSGTDF | |
| signal peptide | TLTISRLEPEDFAVYYCQQYGSSRFTFGPGTKVDIK | |
| | GSTSGSGKPGSGEGSTKGQVQLVQSGAEVKKPGSS | |
| | VKVSCKDSGGTFSSYAISWVRQAPGQGLEWMGGI | |
| | IPIFGTTNYAQQFQGRVTITADESTSTAYMELSSLR | |
| | SEDTAVYYCAREAVAADWLDPWGQGTLVTVSSF | |
| | VPVFLPAKPTTTPAPRPPTPAPTIASQPLSLRPEACR | |
| | PAAGGAVHTRGLDFACDIYIWAPLAGTCGVLLLSL | |
| | VITLYCNHRNRSKRSRLLHSDYMNMTPRRPGPTR | |
| | KHYQPYAPPRDFAAYRSRVKFSRSADAPAYQQGQ | |
| | NQLYNELNLGRREEYDVLDKRRGRDPEMGGKPR | |
| | RKNPQEGLYNELQKDKMAEAYSEIGMKGERRRG | |
| | KGHDGLYQGLSTATKDTYDALHMQALPPR | |
| CD8a transmembrane | IYIWAPLAGTCGVLLLSLVIT | 11 |
| domain | | |
| CD28 transmembrane | FWVLVVVGGVLACYSLLVTVAFIIFWV | 12 |
| domain | | |
| Full-length Hu19- | MALPVTALLLPLALLLHAARPEIVLTQSPGTLSLSP | 13 |
| CD828Z CAR | GERATLSCRASQSVSSSYLAWYQQKPGQAPRLLIY | |
| | GASSRATGIPDRESGSGSGTDFTLTISRLEPEDFAVY | |
| | YCQQYGSSRFTFGPGTKVDIKGSTSGSGKPGSGEG | |
| | STKGQVQLVQSGAEVKKPGSSVKVSCKDSGGTFS | |
| | SYAISWVRQAPGQGLEWMGGIIPIFGTTNYAQQFQ | |
| | GRVTITADESTSTAYMELSSLRSEDTAVYYCAREA | |
| | VAADWLDPWGQGTLVTVSSFVPVFLPAKPTTTPA | |
| | PRPPTPAPTIASQPLSLRPEACRPAAGGAVHTRGLD | |
| | FACDIYIWAPLAGTCGVLLLSLVITLYCNHRNRSK | |
| | RSRLLHSDYMNMTPRRPGPTRKHYQPYAPPRDFA | |
| | AYRSRVKFSRSADAPAYQQGQNQLYNELNLGRRE | |
| | EYDVLDKRRGRDPEMGGKPRRKNPQEGLYNELQ | |
| | KDKMAEAYSEIGMKGERRRGKGHDGLYQGLSTA | |
| | TKDTYDALHMQALPPR | |
| Full-length Hu19- | ATGGCCCTGCCTGTGACAGCTCTGCTGCTGCCCC | 14 |
| CD828Z Chimeric | TGGCCCTGCTGCTGCATGCCGCCAGACCTGAGA | |
| Antigen Receptor- | TCGTGCTGACCCAGTCTCCCGGCACCCTGTCTCT | |
| nucleic acid sequence | CAGCCCAGGAGAGAGAGCCACCCTGAGCTGCAG | |
| | AGCCAGCCAGAGCGTGTCCAGCAGCTACCTGGC | |
| | CTGGTATCAGCAGAAGCCCGGACAGGCCCCCAG | |
| | ACTGCTGATCTACGGCGCCAGCTCTAGAGCCAC | |
| | CGGCATCCCCGACAGATTCAGCGGCAGCGGCAG | |
| | TGGCACCGACTTCACCCTGACCATCAGCAGACT | |
| | GGAACCCGAGGACTTCGCCGTGTACTACTGCCA | |
| | GCAGTACGGCAGCAGCCGGTTCACCTTCGGCCC | |
| | TGGCACCAAGGTGGACATCAAGGGCAGCACCTC | |
| | CGGCAGCGGCAAGCCTGGCTCTGGCGAGGGCTC | |
| | TACCAAGGGCCAGGTGCAGCTGGTGCAGTCTGG | |
| | CGCCGAAGTGAAGAAACCCGGCTCTAGCGTGAA | |
| | GGTGTCCTGCAAGGACAGCGGCGGCACCTTCAG | |
| | CAGCTACGCCATCAGCTGGGTGCGCCAGGCCCC | |
| | AGGACAGGGGCTGGAATGGATGGGCGGCATCAT | |
| | CCCCATCTTCGGCACCACCAACTACGCCCAGCA | |
| | GTTCCAGGGCAGAGTGACCATCACCGCCGACGA | |
| | GAGCACCAGCACCGCCTACATGGAACTGAGCAG | |
| | CCTGCGGAGCGAGGACACAGCCGTGTATTACTG | |
| | TGCCCGCGAGGCCGTGGCCGCCGACTGGCTGGA | |
| | TCCTTGGGGACAGGGCACCCTGGTGACAGTGTC | |
| | CAGCTTCGTGCCCGTGTTCCTGCCCGCCAAGCCT | |
| | ACCACCACCCCTGCCCCTAGACCTCCCACCCCAG | |
| | CCCCAACAATCGCCAGCCAGCCTCTGTCCCTGCG | |
| | GCCCGAAGCCTGTAGACCTGCTGCCGGCGGAGC | |
| | CGTGCACACCAGAGGCCTGGACTTCGCCTGCGA | |
| | TATCTACATCTGGGCCCCTCTGGCCGGCACCTGT | |
| | GGCGTGCTGCTGCTGAGCCTGGTGATCACCCTGT | |
| | ACTGCAACCACCGGAACAGAAGCAAGCGGAGC | |
| | CGGCTGCTGCACAGCGACTACATGAACATGACC | |
| | CCAAGACGGCCTGGCCCCACCCGGAAGCACTAC | |
| | CAGCCTTACGCCCCTCCCAGAGACTTCGCCGCCT | |
| | ACCGGTCCAGAGTGAAGTTCAGCAGATCCGCCG | |
| | ACGCCCCTGCCTACCAGCAGGGACAGAACCAGC | |
| | TGTACAACGAGCTGAACCTGGGCAGACGGGAAG | |
| | AGTACGACGTGCTGGACAAGCGGAGAGGCCGG | |
| | GACCCCGAGATGGGCGGAAAGCCCAGACGGAA | |
| | GAACCCCCAGGAAGGCCTGTATAACGAACTGCA | |
| | GAAAGACAAGATGGCCGAGGCCTACAGCGAGA | |
| | TCGGCATGAAGGGCGAGCGGAGGCGCGGCAAG | |
| | GGCCACGATGGCCTGTACCAGGGCCTGAGCACC | |
| | GCCACCAAGGACACCTACGACGCCCTGCACATG | |
| | CAGGCCCTGCCCCCCAGATGA | |
| CD8a signal peptide | MALPVTALLLPLALLLHAARP | 15 |
| CD8a signal peptide- | ATGGCCCTGCCTGTGACAGCTCTGCTGCTGCCCC | 16 |
| nucleic acid sequence | TGGCCCTGCTGCTGCATGCCGCCAGACCT | |
| Antigen binding domain | EIVLTQSPGTLSLSPGERATLSCRASQSVSSSYLAW | 17 |
| with scFv 47G4 | YQQKPGQAPRLLIYGASSRATGIPDRFSGSGSGTDF | |
| | TLTISRLEPEDFAVYYCQQYGSSRFTFGPGTKVDIK | |
| | GSTSGSGKPGSGEGSTKGQVQLVQSGAEVKKPGSS | |
| | VKVSCKDSGGTFSSYAISWVRQAPGQGLEWMGGI | |
| | IPIFGTTNYAQQFQGRVTITADESTSTAYMELSSLR | |
| | SEDTAVYYCAREAVAADWLDPWGQGTLVTVSS | |
| Antigen binding domain | GAGATCGTGCTGACCCAGTCTCCCGGCACCCTGT | 18 |
| with scFv 47G4- | CTCTCAGCCCAGGAGAGAGAGCCACCCTGAGCT | |
| nucleic acid sequence | GCAGAGCCAGCCAGAGCGTGTCCAGCAGCTACC | |
| | TGGCCTGGTATCAGCAGAAGCCCGGACAGGCCC | |
| | CCAGACTGCTGATCTACGGCGCCAGCTCTAGAG | |
| | CCACCGGCATCCCCGACAGATTCAGCGGCAGCG | |
| | GCAGTGGCACCGACTTCACCCTGACCATCAGCA | |
| | GACTGGAACCCGAGGACTTCGCCGTGTACTACT | |
| | GCCAGCAGTACGGCAGCAGCCGGTTCACCTTCG | |
| | GCCCTGGCACCAAGGTGGACATCAAGGGCAGCA | |
| | CCTCCGGCAGCGGCAAGCCTGGCTCTGGCGAGG | |
| | GCTCTACCAAGGGCCAGGTGCAGCTGGTGCAGT | |
| | CTGGCGCCGAAGTGAAGAAACCCGGCTCTAGCG | |
| | TGAAGGTGTCCTGCAAGGACAGCGGCGGCACCT | |
| | TCAGCAGCTACGCCATCAGCTGGGTGCGCCAGG | |
| | CCCCAGGACAGGGGCTGGAATGGATGGGCGGCA | |
| | TCATCCCCATCTTCGGCACCACCAACTACGCCCA | |
| | GCAGTTCCAGGGCAGAGTGACCATCACCGCCGA | |
| | CGAGAGCACCAGCACCGCCTACATGGAACTGAG | |
| | CAGCCTGCGGAGCGAGGACACAGCCGTGTATTA | |
| | CTGTGCCCGCGAGGCCGTGGCCGCCGACTGGCT | |
| | GGATCCTTGGGGACAGGGCACCCTGGTGACAGT | |
| | GTCCAGC | |
| CD8a hinge and | FVPVFLPAKPTTTPAPRPPTPAPTIASQPLSLR | 19 |
| transmembrane domain | PEACRPAAGGAVHTRGLDFACDIYIWAPLAGTC | |
| | GVLLLSLVITLYCNHRN | |
| CD8a hinge and | TTCGTGCCCGTGTTCCTGCCCGCCAAGCCTACCA | 20 |
| transmembrane domain | CCACCCCTGCCCCTAGACCTCCCACCCCAGCCCC | |
| -nucleic acid sequence | AACAATCGCCAGCCAGCCTCTGTCCCTGCGGCC | |
| | CGAAGCCTGTAGACCTGCTGCCGGCGGAGCCGT | |
| | GCACACCAGAGGCCTGGACTTCGCCTGCGATAT | |
| | CTACATCTGGGCCCCTCTGGCCGGCACCTGTGGC | |
| | GTGCTGCTGCTGAGCCTGGTGATCACCCTGTACT | |
| | GCAACCACCGGAAC | |
| CD28 intracellular | RSKRSRLLHSDYMNMTPRRPGPTRKHYQPYAPPR | 21 |
| domain fragment | DFAAYRS | |
| CD28 intracellular | AGAAGCAAGCGGAGCCGGCTGCTGCACAGCGAC | 22 |
| domain fragment- | TACATGAACATGACCCCAAGACGGCCTGGCCCC | |
| nucleic acid sequence | ACCCGGAAGCACTACCAGCCTTACGCCCCTCCC | |
| | AGAGACTTCGCCGCCTACCGGTCC | |
| CD3 zeta intracellular | RVKFSRSADAPAYQQGQNQLYNELNLGRREEYDV | 23 |
| domain fragment | LDKRRGRDPEMGGKPRRKNPQEGLYNELQKDKM | |
| | AEAYSEIGMKGERRRGKGHDGLYQGLSTATKDTY | |
| | DALHMQALPPR | |
| CD3 zeta intracellular | AGAGTGAAGTTCAGCAGATCCGCCGACGCCCCT | 24 |
| domain fragment- | GCCTACCAGCAGGGACAGAACCAGCTGTACAAC | |
| nucleic acid sequence | GAGCTGAACCTGGGCAGACGGGAAGAGTACGA | |
| | CGTGCTGGACAAGCGGAGAGGCCGGGACCCCGA | |
| | GATGGGCGGAAAGCCCAGACGGAAGAACCCCC | |
| | AGGAAGGCCTGTATAACGAACTGCAGAAAGACA | |
| | AGATGGCCGAGGCCTACAGCGAGATCGGCATGA | |
| | AGGGCGAGCGGAGGCGCGGCAAGGGCCACGAT | |
| | GGCCTGTACCAGGGCCTGAGCACCGCCACCAAG | |
| | GACACCTACGACGCCCTGCACATGCAGGCCCTG | |
| | CCCCCCAGA | |
| Hu19-CD828Z Heavy | GGTFSSY | 25 |
| Chain CDR1 (Chothia) | | |
| Hu19-CD828Z Heavy | IPIFGT | 26 |
| Chain CDR2 (Chothia) | | |
| Hinge-Transmembrane- | FVPVFLPAKPTTTPAPRPPTPAPTIASQPLSLRPEA | 27 |
| Intracellular | CRPAAGGAVHTRGLDFACDIYIWAPLAGTCGVLLLS | |
| portion of | LVITLYCNHRNRSKRSRLLHSDYMNMTPRRPGPTR | |
| Hu19-CD828Z CAR | KHYQPYAPPRDFAAYRSRVKFSRSADAPAYQQGQ | |
| | NQLYNELNLGRREEYDVLDKRRGRDPEMGGKPR | |
| | RKNPQEGLYNELQKDKMAEAYSEIGMKGERRRG | |
| | KGHDGLYQGLSTATKDTYDALHMQALPPR | |
| CD8a hinge domain | FVPVFLPAKPTTTPAPRPPTPAPTIASQPLSLR | 28 |
| | PEACRPAAGGAVHTRGLDFACD | |
| Anti-CD19 Antibody 1 | EVQLVQSGAEVKKPGESLKISCKGSGYSFTNSWIG | 29 |
| VH | WVRQMPGKGLEWMGLIYPDDSDTRYSPSFQGQV | |
| | TISADSAINTAYLQWSSLKASDTAMYYCARQSTYI | |
| | YGGYYDTWGQGTLVTVSS | |
| Anti-CD19 Antibody 1 | QSVVTQPPSVSGAPGQRVTISCTGSSSNIGAGYDV | 30 |
| VL | HWYQQLPGTAPKLLIYENTNRPSGVPDRESGSKSG | |
| | TSASLAITGLQAEDEADYYCQSYDSSLSGWRVFGG | |
| | GTKLTVLG | |
| Anti-CD19 Antibody 2 | EVQLVQSGAEVKKPGESLKISCKGSGYSFTSYWIG | 31 |
| VH | WVRQMPGKGLEWMGIIYPGDSDTRYSPSFQGQVT | |
| | ISADKSISTAYLQWSSLKASDTAMYYCARLSYSWS | |
| | SWYWDFWGQGTLVTVSS | |
| Anti-CD19 Antibody 2 | QAVLTQPPSVSEAPRQRVTISCSGSSSNIGNNAVSW | 32 |
| VL | YQQLPGKAPKLLIYYDDLLPSGVSDRESGSKSGTS | |
| | ASLAISGLQSEDEADYYCAAWDDSLNGWVFGGGT | |
| | KVTVLG | |
| Anti-CD19 FMC63 VH | EVKLQESGPGLVAPSQSLSVTCTVSGVSLPDYGVS | 33 |
| | WIRQPPRKGLEWLGVIWGSETTYYNSALKSRLTII | |
| | KDNSKSQVFLKMNSLQTDDTAIYYCAKHYYYGGS | |
| | YAMDYWGQGTSVTVSS | |
| Anti-CD19 FMC63 VL | DIQMTQTTSSLSASLGDRVTISCRASQDISKYLNW | 34 |
| | YQQKPDGTVKLLIYHTSRLHSGVPSRFSGSGSGTD | |
| | YSLTISNLEQEDIATYFCQQGNTLPYTFGGGTKLEI | |
| | T | |
| Anti-CD19 Antibody 1 | GYSFTNSW | 35 |
| Heavy Chain CDR1 | | |
| Anti-CD19 Antibody 1 | IYPDDSDT | 36 |
| Heavy Chain CDR2 | | |
| Anti-CD19 Antibody 1 | ARQSTYIYGGYYDT | 37 |
| Heavy Chain CDR3 | | |
| Anti-CD19 Antibody 1 | SSNIGAGYD | 38 |
| Light Chain CDR1 | | |
| Anti-CD19 Antibody 1 | ENT | 39 |
| Light Chain CDR2 | | |
| Anti-CD19 Antibody 1 | QSYDSSLSGWRV | 40 |
| Light Chain CDR3 | | |
| Anti-CD19 Antibody 2 | GYSFTSYW | 41 |
| Heavy Chain CDR1 | | |
| Anti-CD19 Antibody 2 | IYPGDSDT | 42 |
| Heavy Chain CDR2 | | |
| Anti-CD19 Antibody 2 | ARLSYSWSSWYWDF | 43 |
| Heavy Chain CDR3 | | |
| Anti-CD19 Antibody 2 | SSNIGNNA | 44 |
| Light Chain CDR1 | | |
| Anti-CD19 Antibody 2 | YDD | 45 |
| Light Chain CDR2 | | |
| Anti-CD19 Antibody 2 | AAWDDSLNGWV | 46 |
| Light Chain CDR3 | | |
| Anti-CD19 FMC63 | GVSLPDY | 47 |
| Heavy Chain CDR1 | | |
| Anti-CD19 FMC63 | WGSET | 48 |
| Heavy Chain CDR2 | | |
| Anti-CD19 FMC63 | HYYYGGSYAMDY | 49 |
| Heavy Chain CDR3 | | |
| Anti-CD19 FMC63 | RASQDISKYLN | 50 |
| Light Chain CDR1 | | |
| Anti-CD19 FMC63 | HTSRLHS | 51 |
| Light Chain CDR2 | | |
| Anti-CD19 FMC63 | QQGNTLPYT | 52 |
| Light Chain CDR3 | | |
| Signaling sequence | YNMN | 53 |
| motif | | |

What is claimed is:

1. A method of treating lupus nephritis, the method comprising administering to a subject in need thereof a therapeutically effective amount of T cells that comprises a vector comprising a nucleic acid encoding a chimeric antigen receptor (CAR), wherein the CAR comprises, from N-terminus to C-terminus:
(a) an antigen-binding fragment of an anti-CD19 antibody;
(b) a transmembrane domain; and
(c) an intracellular T cell signaling domain from human CD3ζ.

2. The method of claim 1, wherein the lupus nephritis is Class III or Class IV lupus nephritis.

3. The method of claim 1 or 2, wherein the anti-CD19 antibody is a human antibody.

4. The method of any one of claims 1-3, wherein the antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 25, 26, and 3, respectively, and the light chain variable domain comprising CDR1, CDR2, and CDR3 amino acid sequences of SEQ ID NOs: 4, 5, and 6, respectively.

5. The method of any one of claims 1-3, wherein the antigen-binding fragment of the anti-CD19 antibody comprises a heavy chain variable domain and a light chain variable domain, the heavy chain variable domain comprising CDR1, CDR2, and CDR3 amino acid sequences of SEQ ID NOs: 1, 2, and 3, respectively, and the light chain variable domain comprising CDR1, CDR2, and CDR3 amino acid sequences of SEQ ID NOs: 4, 5, and 6, respectively.

6. The method of claim 4 or 5, wherein the heavy chain variable domain comprises an amino acid sequence at least 90% identical to SEQ ID NO: 7, and the light chain variable domain comprises an amino acid sequence at least 90% identical to SEQ ID NO: 8.

7. The method of claim 6, wherein the heavy chain variable domain comprises the amino acid sequence of SEQ ID NO: 7, and the light chain variable domain comprises the amino acid sequence of SEQ ID NO: 8.

8. The method of claim 7, wherein the antigen-binding fragment of the anti-CD19 antibody comprises the amino acid sequence of SEQ ID NO: 17.

9. The method of any one of claims 1-8, wherein the transmembrane domain is from human CD8.

10. The method of any one of claims 1-9, wherein the transmembrane domain comprises an amino acid sequence at least 90% identical to SEQ ID NO: 11.

11. The method of claim 10, wherein the transmembrane domain comprises the amino acid sequence of SEQ ID NO: 11.

12. The method of any one of claims 1-11, wherein the intracellular T cell signaling domain from human CD3ζ comprises an amino acid sequence at least 90% identical to SEQ ID NO: 23.

13. The method of claim 12, wherein the intracellular T cell signaling domain from human CD3ζ comprises the amino acid sequence of SEQ ID NO: 23.

14. The method of any one of claims 1-13, wherein the CAR further comprises an intracellular T cell signaling domain from human CD28.

15. The method of claim 14, wherein the intracellular T cell signaling domain from human CD28 comprises the amino acid sequence of SEQ ID NO: 21.

16. The method of any one of claims 1-13, wherein the CAR does not comprise an intracellular T cell signaling domain from 4-1BB.

17. The method of any one of claims 1-16, wherein the CAR comprises an amino acid sequence of SEQ ID NO: 10 or 13.

18. The method of any one of claims 1-17, wherein the vector is a lentivirus vector.

19. The method of any one of claims 1-18, wherein the vector further comprises a murine stem cell virus (MSCV) U3 promoter operably linked to the nucleic acid.

20. The method of any one of claims 1-19, wherein at least 10% of the T cells express the CAR.

21. The method of any one of claims 1-20, wherein the T cells comprise at least 10% of CD8⁺ cytotoxic T cells.

22. The method of any one of claims 1-21, wherein the T cells comprise at least 10% of CD4⁺ helper T cells.

23. The method of any one of claims 1-22, wherein the lupus nephritis is active, biopsy-proven, proliferative lupus nephritis of Class III or Class IV according to the 2018 ISN/RPS criteria.

24. The method of claim 23, wherein the lupus nephritis is active, biopsy-proven, proliferative lupus nephritis of Class III according to the 2018 ISN/RPS criteria.

25. The method of claim 23, wherein the lupus nephritis is active, biopsy-proven, proliferative lupus nephritis of Class IV according to the 2018 ISN/RPS criteria.

26. The method of any one of claims 1-25, wherein the therapeutically effective dose is in a range of about 5×10⁷ to 1×10⁸ of the T cells.

27. The method of any one of claims 1-25, wherein the therapeutically effective dose is about 5×10⁷ of the T cells.

28. The method of any one of claims 1-25, wherein the therapeutically effective dose is about 1×10⁸ of the T cells.

29. The method of any one of claims 1-28, wherein the T cells are administered by intravenous infusion.

30. The method of any one of claims 1-29, wherein the subject receives a single dose of the T cells.

31. The method of any one of claims 1-30, wherein the subject has received a lymphodepletion treatment.

32. The method of any one of claims 1-31, wherein the subject has received a minimal lymphodepletion treatment resulting in about 50% reduction of lymphocytes in the subject as compared to the amount of lymphocytes in the subject prior to the minimal lymphodepletion treatment.


SEQUENCE LISTING (53 sequences)

SEQ ID NO: 1 - protein, 5 aa, synthetic construct
SYAIS

SEQ ID NO: 2 - protein, 17 aa, synthetic construct
GIIPIFGTTNYAQQFQG

SEQ ID NO: 3 - protein, 10 aa, synthetic construct
EAVAADWLDP

SEQ ID NO: 4 - protein, 12 aa, synthetic construct
RASQSVSSSYLA

SEQ ID NO: 5 - protein, 7 aa, synthetic construct
GASSRAT

SEQ ID NO: 6 - protein, 9 aa, synthetic construct
QQYGSSRFT

SEQ ID NO: 7 - protein, 119 aa, synthetic construct
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 8 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 9 - protein, 18 aa, synthetic construct
GSTSGSGKPGSGEGSTKG

SEQ ID NO: 10 - protein, 481 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 21 aa, synthetic construct
IYIWAPLAGTCGVLLLSLVIT

SEQ ID NO: 12 - protein, 27 aa, synthetic construct
FWVLVVVGGVLACYSLLVTVAFIIFWV

SEQ ID NO: 13 - protein, 502 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 1509 bp, synthetic construct
  source 1..1509: mol_type = other DNA
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatga

SEQ ID NO: 15 - protein, 21 aa, synthetic construct
MALPVTALLLPLALLLHAARP

SEQ ID NO: 16 - DNA, 63 bp, synthetic construct
  source 1..63: mol_type = other DNA
atggccctgcctgtgacagctctgctgctgcccctggccctgctgctgcatgccgccagacct

SEQ ID NO: 17 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 18 - DNA, 735 bp, synthetic construct
  source 1..735: mol_type = other DNA
gagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagc

SEQ ID NO: 19 - protein, 83 aa, synthetic construct
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 20 - DNA, 249 bp, synthetic construct
  source 1..249: mol_type = other DNA
ttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaac

SEQ ID NO: 21 - protein, 41 aa, synthetic construct
RSKRSRLLHSDYMNMTPRRPGPTRKHYQPYAPPRDFAAYRS

SEQ ID NO: 22 - DNA, 123 bp, synthetic construct
  source 1..123: mol_type = other DNA
agaagcaagcggagccggctgctgcacagcgactacatgaacatgaccccaagacggcctggccccacccggaagcactaccagccttacgcccctcccagagacttcgccgcctaccggtcc

SEQ ID NO: 23 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 336 bp, synthetic construct
  source 1..336: mol_type = other DNA
agagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 25 - protein, 7 aa, synthetic construct
GGTFSSY

SEQ ID NO: 26 - protein, 6 aa, synthetic construct
IPIFGT

SEQ ID NO: 27 - protein, 236 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 55 aa, synthetic construct
FVPVFLPAKPTTTPAPRPPTPAPTIASQPLSLRPEACRPAAGGAVHTRGLDFACD

SEQ ID NO: 29 - protein, 121 aa, synthetic construct
EVQLVQSGAEVKKPGESLKISCKGSGYSFTNSWIGWVRQMPGKGLEWMGLIYPDDSDTRYSPSFQGQVTISADSAINTAYLQWSSLKASDTAMYYCARQSTYIYGGYYDTWGQGTLVTVSS

SEQ ID NO: 30 - protein, 113 aa, synthetic construct
QSVVTQPPSVSGAPGQRVTISCTGSSSNIGAGYDVHWYQQLPGTAPKLLIYENTNRPSGVPDRFSGSKSGTSASLAITGLQAEDEADYYCQSYDSSLSGWRVFGGGTKLTVLG

SEQ ID NO: 31 - protein, 121 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSS

SEQ ID NO: 32 - protein, 111 aa, synthetic construct
QAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLG

SEQ ID NO: 33 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 34 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 35 - protein, 8 aa, synthetic construct
GYSFTNSW

SEQ ID NO: 36 - protein, 8 aa, synthetic construct
IYPDDSDT

SEQ ID NO: 37 - protein, 14 aa, synthetic construct
ARQSTYIYGGYYDT

SEQ ID NO: 38 - protein, 9 aa, synthetic construct
SSNIGAGYD

SEQ ID NO: 39 - skipped

SEQ ID NO: 40 - protein, 12 aa, synthetic construct
QSYDSSLSGWRV

SEQ ID NO: 41 - protein, 8 aa, synthetic construct
GYSFTSYW

SEQ ID NO: 42 - protein, 8 aa, synthetic construct
IYPGDSDT

SEQ ID NO: 43 - protein, 14 aa, synthetic construct
ARLSYSWSSWYWDF

SEQ ID NO: 44 - protein, 8 aa, synthetic construct
SSNIGNNA

SEQ ID NO: 45 - skipped

SEQ ID NO: 46 - protein, 11 aa, synthetic construct
AAWDDSLNGWV

SEQ ID NO: 47 - protein, 7 aa, synthetic construct
GVSLPDY

SEQ ID NO: 48 - protein, 5 aa, synthetic construct
WGSET

SEQ ID NO: 49 - protein, 12 aa, synthetic construct
HYYYGGSYAMDY

SEQ ID NO: 50 - protein, 11 aa, synthetic construct
RASQDISKYLN

SEQ ID NO: 51 - protein, 7 aa, synthetic construct
HTSRLHS

SEQ ID NO: 52 - protein, 9 aa, synthetic construct
QQGNTLPYT

SEQ ID NO: 53 - protein, 4 aa, synthetic construct
YNMN